(19) United States
(12) Patent Application Publication
(10) Pub. No.: US 2011/0166059 A1
(43) Pub. Date: Jul. 7, 2011

(54) MEANS AND METHODS FOR EVALUATING A THERAPY WITH A P38 MAP KINASE INHIBITOR

(76) Inventor: Dorothee Viemann, Muenster (DE)

(21) Appl. No.: 13/063,730

(22) PCT Filed: Sep. 14, 2009
(86) PCT No.: PCT/EP09/61840

§ 371 Date: Mar. 11, 2011

Related U.S. Application Data
(60) Provisional application No. 61/108,878, filed on Oct. 28, 2008.

(30) Foreign Application Priority Data
Sep. 12, 2008 (EP) 08105324.1

(51) Int. Cl. A61K 38/00; C12Q 1/68; C07K 14/00

(52) U.S. Cl. 514/1.1; 435/6.1; 530/350

(57) ABSTRACT

The present invention relates to a method for determining whether therapy with a p38 MAP kinase inhibitor is potentially beneficial or potentially contraindicated for a subject suffering from a p38-mediated condition comprising measuring in a sample obtained from the subject the presence of at least one chromatin remodelling gene and/or of at least one pro-inflammatory gene, wherein the treatment is potentially beneficial for the subject, if at least one chromatin remodelling gene is underrepresented and/or at least one pro-inflammatory gene is overrepresented in comparison to a reference sample obtained from a subject not suffering from a p38-mediated condition; or wherein the treatment is potentially contraindicated for the subject, if at least one chromatin remodelling gene is overrepresented and/or at least one pro-inflammatory gene is underrepresented in comparison to a reference sample obtained from a subject not suffering from a p38-mediated condition. The present invention also relates to medical uses and methods of treatment applying a p38 MAP kinase inhibitor for treating a p38-mediated condition in a subject, wherein the patient is amenable to the treatment with the p38 MAP kinase inhibitor, if in the subject at least one chromatin remodelling gene is underrepresented and/or at least one pro-inflammatory gene is overrepresented in comparison to a reference sample obtained from a subject not suffering from a p38-mediated condition. Furthermore, a packaged medicament and a kit are provided comprising a p38 MAP kinase inhibitor or means for determining the presence of at least one chromatin remodelling gene and/or at least one pro-inflammatory gene in a sample from a subject suffering from a p38-mediated condition and instructions for use indicating that a subject suffering from a p38-mediated condition is amenable to the treatment with the p38 MAP kinase inhibitor, if it has been determined whether in said subject at least one chromatin remodelling gene is underrepresented and/or at least one pro-inflammatory gene is overrepresented in comparison to a reference sample obtained from a subject not suffering from a p38-mediated condition.

The present invention relates to a method for determining whether therapy with a p38 MAP kinase inhibitor is potentially beneficial or potentially contraindicated for a subject suffering from a p38-mediated condition comprising measuring in a sample obtained from the subject the presence of at least one chromatin remodelling gene and/or of at least one pro-inflammatory gene, wherein the treatment is potentially beneficial for the subject, if at least one chromatin remodelling gene is underrepresented and/or at least one pro-inflammatory gene is overrepresented in comparison to a reference sample obtained from a subject not suffering from a p38-mediated condition; or wherein the treatment is potentially contraindicated for the subject, if at least one chromatin remodelling gene is overrepresented and/or at least one pro-inflammatory gene is underrepresented in comparison to a reference sample obtained from a subject not suffering from a p38-mediated condition.

The present invention also relates to medical uses and methods of treatment applying a p38 MAP kinase inhibitor for treating a p38-mediated condition in a subject, wherein the patient is amenable to the treatment with the p38 MAP kinase inhibitor, if in the subject at least one chromatin remodelling gene is underrepresented and/or at least one pro-inflammatory gene is overrepresented in comparison to a reference sample obtained from a subject not suffering from a p38-mediated condition.

Furthermore, a packaged medicament and a kit are provided comprising a p38 MAP kinase inhibitor or means for determining the presence of at least one chromatin remodelling gene and/or at least one pro-inflammatory gene in a sample from a subject suffering from a p38-mediated condition and instructions for use indicating that a subject suffering from a p38-mediated condition is amenable to the treatment with the p38 MAP kinase inhibitor, if it has been determined whether in said subject at least one chromatin remodelling gene is underrepresented and/or at least one pro-inflammatory gene is overrepresented in comparison to a reference sample obtained from a subject not suffering from a p38-mediated condition.

Mitogen-activated protein (MAP) kinases (also abbreviated as MAPK) are protein kinases that respond to extracellular stimuli (mitogens) and regulate various cellular activities, such as gene expression, mitosis, differentiation, and cell survival/apoptosis. MAPKs specifically phosphorylate proteins at serine/threonine (S/T) residues. They are thus key enzymes involved in signal transduction and the amplification of cellular responses to stimuli.

MAPKs are normally activated by phosphorylation at two sites, in particular, at a tyrosine (Y) residue and a threonine (T) residue, which are separated from each other by one amino acid. However, also phosphorylation at one of these residues can be sufficient to activate MAPKs. This phosphorylation is done by a MAP kinase kinase (also abbreviated as MAPKK), which is “upstream” in the cascade of a MAPK pathway. Once a MAPK is activated, it accumulates in the nucleus where it phosphorylates transcription factors. Accordingly, a MAPK is thus capable of regulating gene expression.

If a MAPK is dephosphorylated, it becomes inactive, leaves the nucleus and resides in the cytoplasm so as to be again available for being phosphorylated by a MAPKK.

MAPKs can be divided in three groups:

(1) extracellular signal regulated kinases (ERK);

(2) p38 mitogen activated protein kinases (p38 MAPK); and

(3) c-Jun N-terminal kinases (JNK).

As mentioned above, p38 MAPKs are involved in signal transduction pathways triggered by external stimuli such as cytokines, heat shock, radiation, osmotic stress etc. They also play a role in cell differentiation, cell growth and apoptosis. p38 MAP kinases are responsible for phosphorylating and activating transcription factors as well as other kinases, and are themselves activated by physical and chemical stress, pro-inflammatory cytokines and bacterial lipopolysaccharide.

Four isoforms of p38 MAPKs are known, i.e., p38-α (MAPK14), -β (MAPK11), -γ (MAPK12 or ERK6) and -δ (MAPK13 or SAPK4) (Ono et al. (2000) Cellular Signalling 12:1-13).

The p38 group of MAP kinases is a group of MAP kinases which are in particular associated with the onset and progression of inflammation. Early inflammatory events include cytokine release, activation and rapid accumulation of neutrophils with subsequent recruitment of mononuclear cells. p38 MAP kinase plays a central role in regulating a wide range of inflammatory responses in many different cells. Recent studies have shown that a p38 MAP kinase inhibitor [(S)-5-[2-(1-phenylethylamino)pyrimidin-4-yl]-1-methyl-4-(3-trifluoromethylphenyl)-2-(4-piperidiny)imidazole] reduced initial neutrophil recruitment to the lung in a murine model of mild pulmonary inflammation induced by lipopolysaccharide (LPS) (Nick et al. (2000) J. Immunol. 164:2151-2159.) p38 MAP kinase is activated by dual phosphorylation after stimulation by a wide array of extracellular stimuli including physiochemical stress, treatment with lipopolysaccharide (LPS) or E. coli, signal transduction from the Toll-like receptors, as well as, TNF and IL-1 receptors. The products of the p38 phosphorylation mediate the production of inflammatory cytokines, including TNF, IL-1, IL-6, iNOS and cyclooxygenase-2.

The products of p38 phosphorylation have been shown to mediate the production of inflammatory cytokines, including TNF, IL-1α, IL-1β, IL-6, IL-8, IL-18, interferon γ, platelet-activating factor (PAF), macrophage migration inhibitory factor (MIF), and other compounds. Certain other compounds, for example high mobility group protein 1 (HMG-1), are induced during various conditions such as sepsis and can also serve as pro-inflammatory cytokines. Each of these cytokines has been implicated in numerous disease states and conditions. For example, TNF-α is a cytokine produced primarily by activated monocytes and macrophages. Its excessive or unregulated production has been implicated in the pathogenesis of rheumatoid arthritis.

In view of the important role of p38 MAP kinase in various processes including inflammatory responses, the pharmaceutical industry has become highly interested in developing p38 MAP kinase inhibitors for treating, for example, p38-mediated conditions, for example, inflammatory disorders.

Thus, the application of p38 MAP kinase inhibitor is presently proposed and effected as anti-inflammatory agents. However, as is known for other agents in order to avoid side effects or even more severe deleterious effects for a patient, it would be highly desirable to have means and methods available which allow to determine whether or not therapy with a p38 MAP kinase inhibitor is likely to be beneficial for a patient suffering from a p38-mediated condition or is likely to be contraindicated for such a patient.

Hence, there is a need to provide means and methods which allow a more targeted and/or tailored therapy with a p38 MAP kinase inhibitor.

Accordingly, the technical problem underlying the present application is to provide means and methods which allow a more targeted and/or tailored therapy with a p38 MAP kinase inhibitor.

The technical problem is solved by the embodiments which follow.

Thus, in a first aspect the present invention relates to a method for determining whether therapy with a p38 MAP kinase inhibitor is potentially beneficial or potentially contraindicated for a subject suffering from a p38-mediated condition or being at a risk thereof comprising measuring in a sample obtained from the subject the presence of at least one chromatin remodelling gene and/or of at least one pro-inflammatory gene, wherein the treatment is potentially beneficial for the subject, if at least one chromatin remodelling gene is underrepresented and/or at least one pro-inflammatory gene is overrepresented in comparison to a reference sample obtained from a subject not suffering from a p38-mediated condition or not being at a risk thereof; or wherein the treatment is potentially contraindicated for the subject, if at least one chromatin remodelling gene is overrepresented and/or at least one pro-inflammatory gene is underrepresented in comparison to a reference sample obtained from a subject not suffering from a p38-mediated condition.

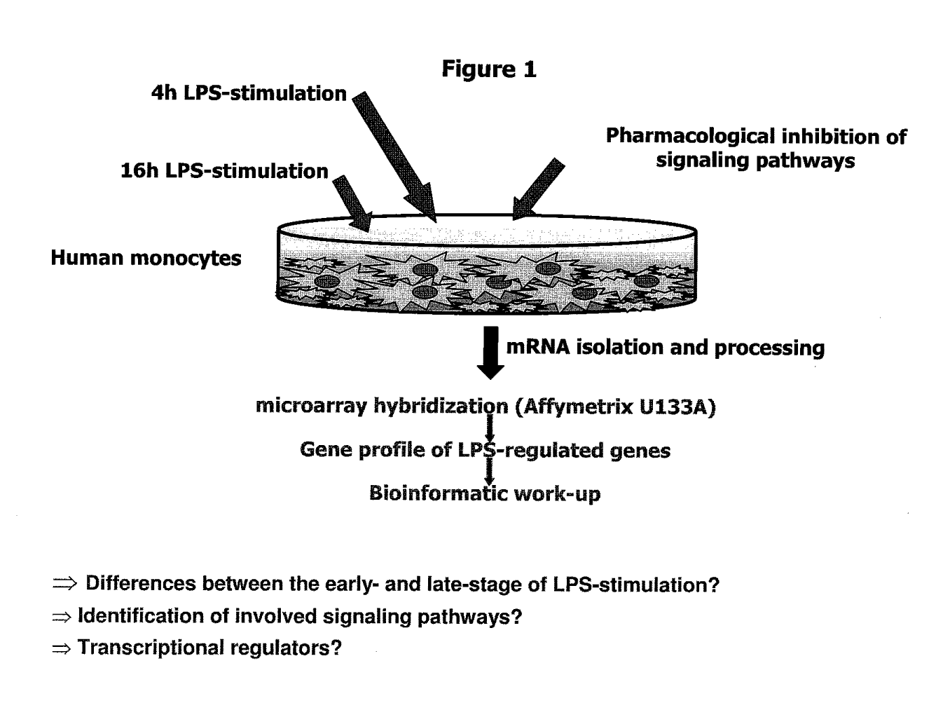

The present inventor has surprisingly found that p38 MAP kinase mediates an anti-inflammatory stage of monocytes after long-term stimulation with lipopolysaccharide (LPS) by epigenetic silencing. Specifically, lipopolysaccharide (LPS) as major constituent of gram-negative bacteria is known to elicit a rapid pro-inflammatory primary response in immune cells. Multiple findings indicate that monocytes next to its crucial function in the early stage of LPS signalling might additionally play an essential role in the resolution of systemic inflammatory responses. The present inventor assumed a differential regulation of gene expression at the transcriptional level that discriminates between pro-inflammatory genes potentially causing tissue damage and antimicrobial effects essential for ongoing host defense. Accordingly, they have chosen a microarray approach to decode involved signalling pathways virtually retrograde via differences between gene expression profiles of monocytes treated short-term and long-term with LPS. Thus, they compared gene expression profiles of short-term and long-term LPS-stimulated monocytes; see FIG. 1.

“Short-term” stimulation with LPS means about 1, 2, 3 or preferably 4 hours. “Long term” stimulation with LPS means at least about 5, 6, 7, 8, 9, 10, 11, 12, 13, 14 or 15 hours, preferably at least 16 hours. LPS treatment of monocytes is a representative model for acute and/or chronic inflammation, since monocytes are in particular involved in inflammation. Inflammation, as is commonly known, can lead to a host of other diseases as described herein. Accordingly, the observations and findings of the present inventor in the monocytes model system provide are generalizable in that, without being bound by theory, may be illustrative for the in vivo situation in subjects (see also Branger et al. in J Immunol. 2002 Apr. 15; 168(8):4070-4077.

Strikingly, the present inventor revealed an anti-inflammatory gene expression program after long-term LPS treatment.

The term “anti-inflammatory gene expression program” or “anti-inflammatory response” means that in cells, preferably cells of the immune system such as T cells, B cells, NK cells, macrophages or monocytes, genes involved in an inflammatory response are down-regulated. Preferably, these genes are down-regulated to about 10, 20, 30, 40, 50, 60, 70, 80, 90 or even 100% when compared to their induced state, i.e., during an inflammatory response.

Functional clustering analysis showed that inflammatory response genes were found to be overrepresented in the group of down-regulated genes in the late stage of LPS-signalling. The down-regulation of inflammatory response genes could be confirmed by RT-PCR.

Remarkably at the late stage of LPS-signalling, chromatin remodelling genes were found to be significantly overrepresented in the group of p38 MAP kinase-dependent genes.

With the aim of finding a key player in the down-regulation of inflammatory response genes and induction of chromatin remodelling genes, the present inventor performed inhibition experiments by using kinase inhibitors to elucidate the pathway which may be responsible for the effects observed.

Blocking different signalling pathways, i.e., ERK kinase, PKC and p38 kinase pathway, revealed p38 MAP kinase to be significantly involved in the regulation of an anti-inflammatory program. The p38 MAP kinase inhibitor applied by the present inventor can inhibit the p38 MAP kinase-α, -β, and -γ isoforms. For example, the inhibitor can be SB202190, i.e., pyridinyl imidazole, 4-(4-fluorophenyl)-2-2(4-hydroxyphenyl)-5-(4-pyridyl)-imidazole (identical to SB202190); see J Biol Chem 1997; 272(48):30122-30128.

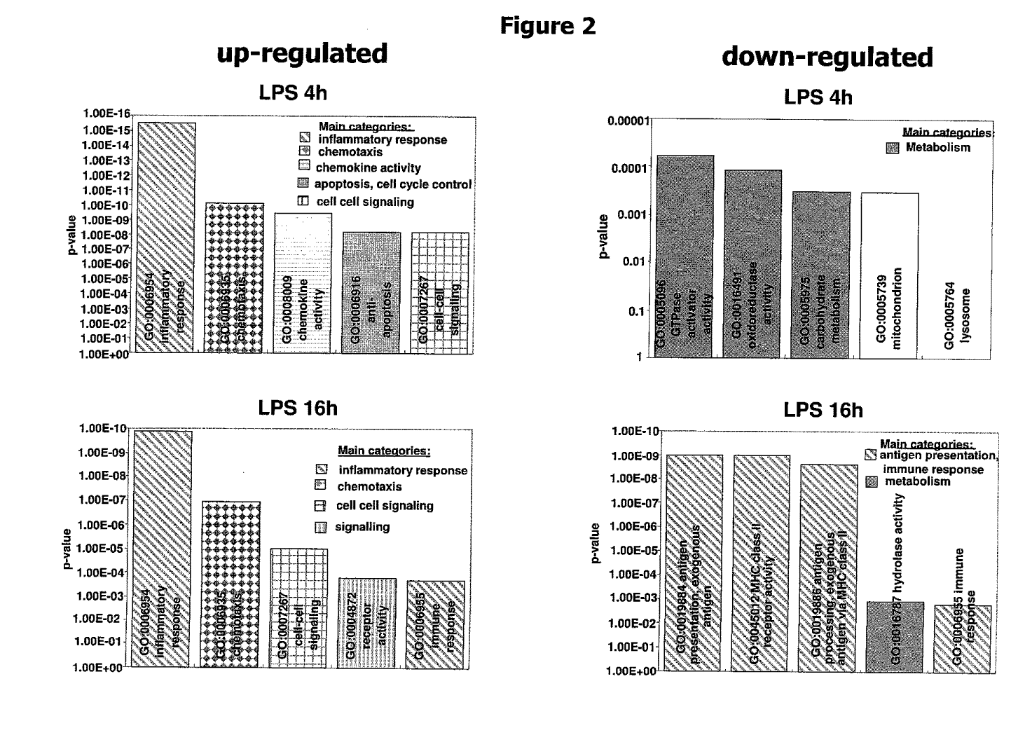

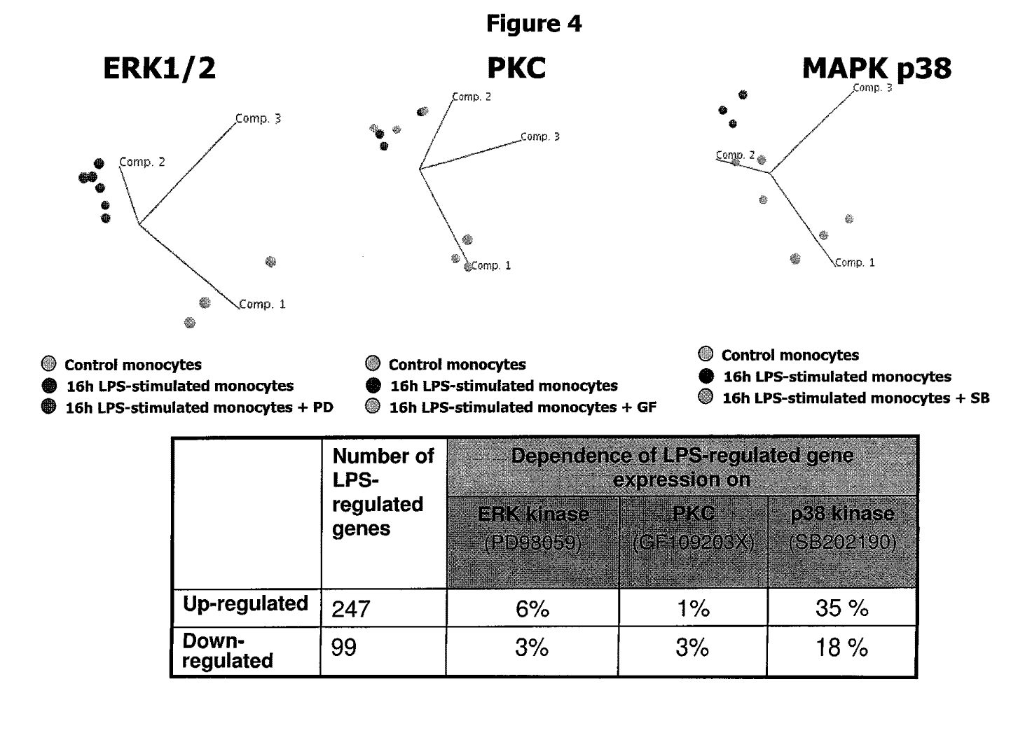

Indeed, it was demonstrated that inhibition of p38 MAP kinase by using the inhibitor SB202190 reversed the down-regulation of inflammatory response genes, which was assumed to be effected by epigenetic silencing, since functional clustering revealed that chromatin remodelling genes and nucleosome assembly genes are overrepresented in p38 MAP kinase dependent genes in the late stage of LPS-signaling; see FIGS. 2, 3, 5 and 10. Inhibition experiments using inhibitors of ERK kinase (PD98059) and PKC (GF109203X) showed that in contrast to ERK1/2 and PKC p38 MAP kinase is specifically and significantly involved in late-stage LPS-induced gene expression changes; see FIG. 4.

Thus, without being bound by theory, the p38 MAP kinase-α, -β, and/or -γ isoforms can mediate epigenetic silencing. Epigenetic silencing is assumed to be directly or indirectly effected by p38 MAP kinase.

Accordingly, to test whether epigenetic modifications cause transcriptional silencing in long-term LPS-treated monocytes and whether this process is p38 MAP kinase dependent, the present inventor also performed chromatin-immunoprecipitation (ChIP).

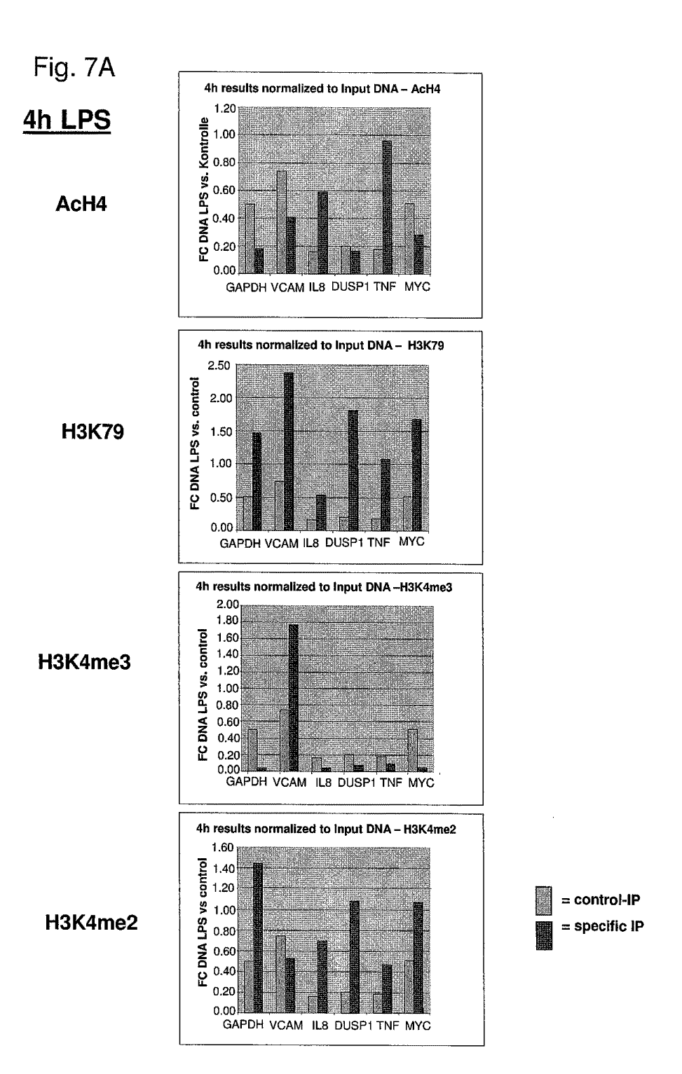

The present inventor observed for pro-inflammatory genes an increased trimethylation of histone 3 (H3K4me3) and methylation of H3K79 as well as increased acetylation of histone 4 (AcH4) after 4 h LPS treatment indicating increased transcription whereas after 16 h of LPS treatment acetylation of histone 4 is decreased; see FIG. 7.

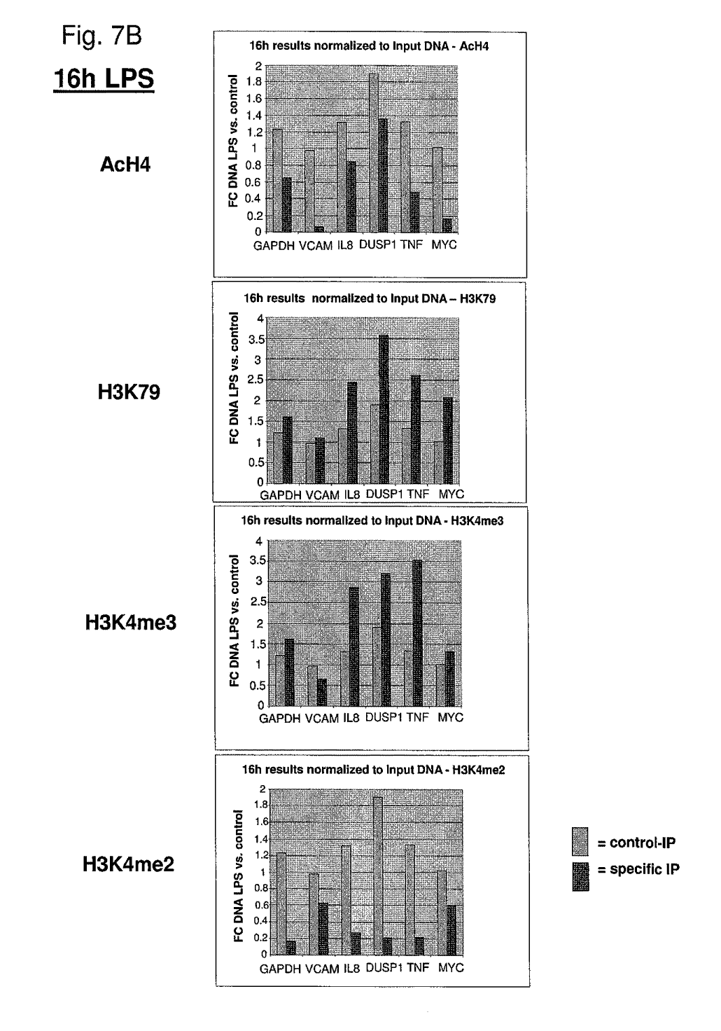

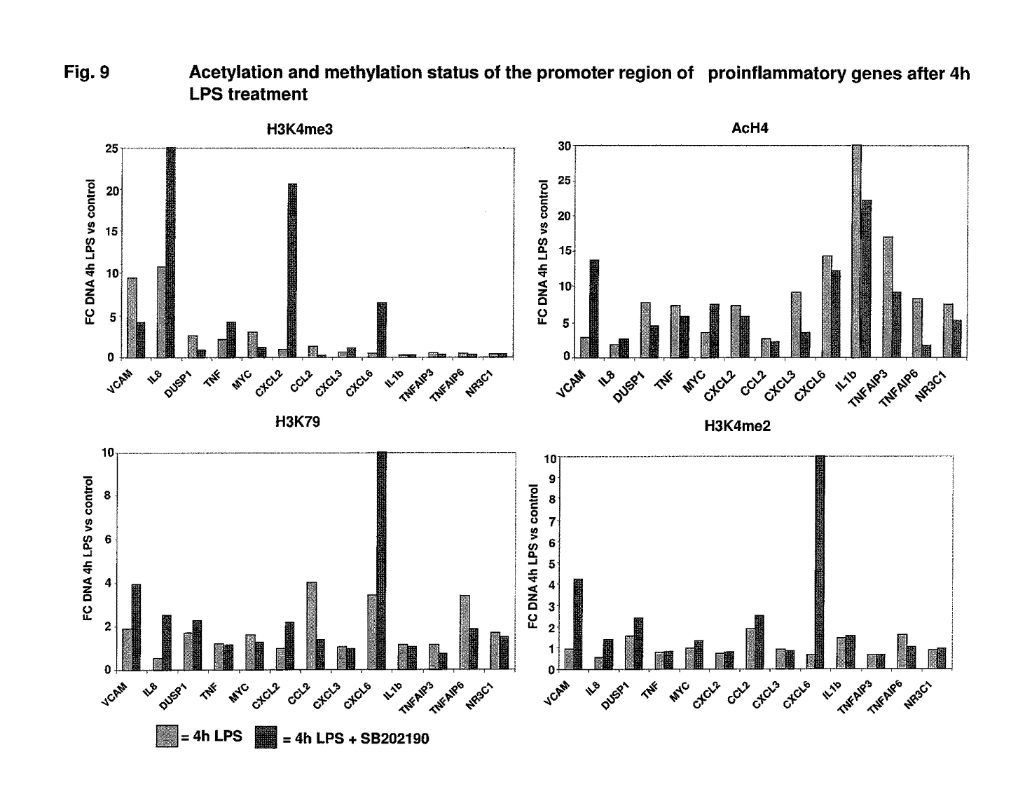

However, ongoing inhibition of p38 MAP kinase by using a p38 MAP kinase inhibitor such as SB 202190 reversed the long-term LPS effect causing a sustained high acetylation of H4 (AcH4) and methylation of K79 (H3K79), both are indicators of high transcriptional activity; see FIGS. 8 and 10. Both, acetylation of H4 as well as methylation of H3K79 are known to be strongly correlated with gene expression (Steger (2008), Mol Cell Biol. 8:2825-2839).

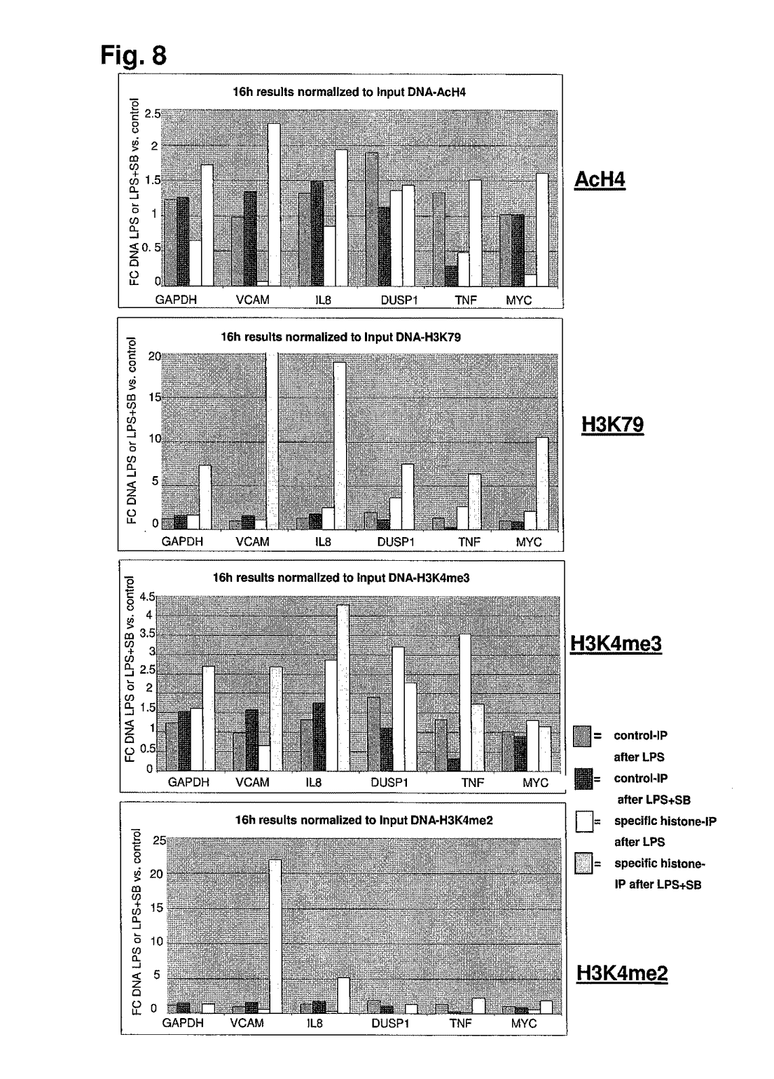

Interestingly, the simultaneous inhibition of p38 MAP kinase during the early stage of LPS treatment does apparently not epigenetically modify pro-inflammatory genes, since the methylation and acetylation of histones at the promoter of pro-inflammatory genes was not decreased in comparison to the control; see FIG. 9. Thus, it may be assumed that p38 MAP kinase plays a dual role. Firstly, it activates expression of, for example, pro-inflammatory genes by, for example, involving transcriptional activators like NF-kB, while, secondly, at a later stage of inflammation p38 MAP kinase de-activates pro-inflammatory genes by, for example, epigenetic modifications of pro-inflammatory genes as shown herein. p38 MAP kinase is assumed to effect epigenetic modifications by way of effecting expression of chromatin remodelling genes encoding, inter alia, histone modifying enzymes such as histone acetylases, histone deacetylases, histone methylases and/or histone demethylases as described in more detail herein.

These results show that p38 MAP kinase contributes to an inflammatory gene expression program and significantly to an anti-inflammatory gene expression program in monocytes after long-term LPS treatment.

In essence, bioinformatic analysis of microarray data obtained by the present inventor in combination with ChIPs revealed a functional clustering of LPS-regulated genes according to gene ontology annotations. Surprisingly, after long-term treatment with LPS inflammatory response genes were found to be overrepresented in the group of down-regulated genes, while chromatin remodeling genes were overrepresented.

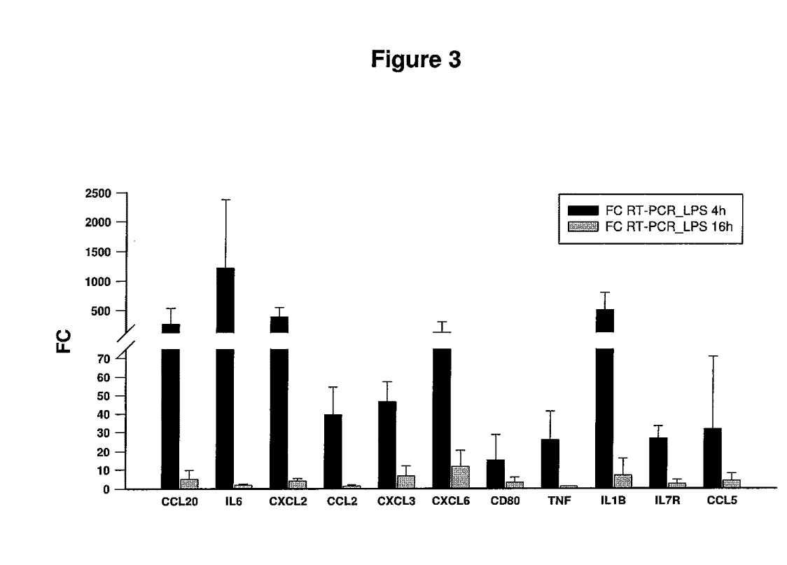

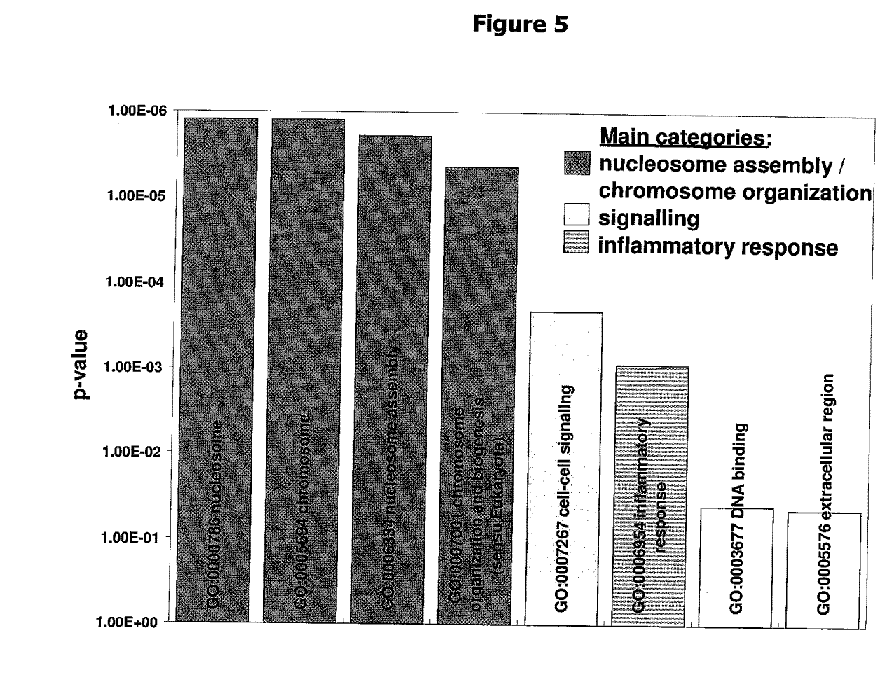

In sum, the present inventor has observed that
p38 MAP kinase is involved in an inflammatory gene expression program in human monocytes in the initial phase of LPS treatment (see FIG. 9);
in human monocytes long-term treatment with LPS causes an anti-inflammatory gene profile (see FIGS. 2 and 3);
in this stage down-regulation of pro-inflammatory genes is significantly dependent on the p38 MAPK pathway (see FIG. 4);
in the group of p38-dependent genes those involved in chromatin remodelling and nucleosome assembly are overrepresented (see FIG. 5);
ChIP-analyses of long-term LPS-induced monocytes revealed an acetylation and methylation pattern that is accompanied by decreased transcription of pro-inflammatory genes and was dependent on p38 MAPK pathway (see FIGS. 7 and 8); and
results indicate that in ongoing infections p38 MAP kinase might mediate epigenetic silencing of LPS-induced pro-inflammatory genes.

From these observations, it can be concluded that p38 MAP kinase is in the early stage of LPS-signaling involved in the induction of inflammatory response genes, while in the late stage of LPS-signaling, it is involved in epigenetic silencing, which, in turn, down-regulates inflammatory response genes. In fact, Branger (2002), J. Immunol. 168:4070-4077 has shown that the p38 mitogen-activated protein kinase (MAPK) participates in intracellular signaling cascades resulting in inflammatory responses. In particular, after LPS administration to human volunteers cytokines such as TNF, IL-6 or IL-10 significantly increased after about 2-4 hours in the control group. The group that received the p38 MAPK inhibitor BIRB 796 BS did not show a significant increase in these cytokines. The in vivo data show that p38 MAP kinase is involved in the activation of pro-inflammatory cytokines. Accordingly, it was suggested that the anti-inflammatory potential of an oral p38 MAPK inhibitor in humans in vivo may provide a new therapeutic option in the treatment of inflammatory diseases.

However, as has been observed by the present inventor it is important to know the “status” of a subject suffering from a p38-mediated condition. Status means the expression level or amount of a pro-inflammatory gene/protein and/or of a chromatin remodelling gene/protein. Namely, if the status of a pro-inflammatory gene/protein is high, i.e., above the level of a healthy subject, and/or the status of a chromatin remodelling gene/protein may be low, i.e, below or equal to the level of a healthy subject, the subject may be amenable to a treatment with a p38 MAP kinase inhibitor.

Likewise, if the status of a pro-inflammatory gene/protein is low, i.e., below or equal to the level of a healthy subject, and/or the status of a chromatin remodelling gene/protein may be high, i.e, higher than the level of a healthy subject, the subject may not be amenable to a treatment with a p38 MAP kinase inhibitor.

The status of a subject as regards the expression level or amount of a pro-inflammatory gene/protein and/or of a chromatin remodelling gene/protein can be determined by determining the expression level or amount of a pro-inflammatory gene/protein and/or of a chromatin remodelling gene/protein as described herein.

Accordingly, the present invention shows that it is of utmost importance to first determine the status of a subject as regards the expression level or amount of a pro-inflammatory gene/protein and/or of a chromatin remodelling gene/protein before a p38 MAP kinase inhibitor is administered for a therapy of p38-mediated conditions. In order to set the status of a subject suffering from a p38-mediated condition or being at a risk thereof in relation, the expression level or amount of a pro-inflammatory gene/protein and/or of a chromatin remodelling gene/protein is to be compared to either the expression level or amount of a pro-inflammatory gene/protein and/or of a chromatin remodelling gene/protein of a healthy subject and/or is to be compared to said subject suffering from a p38-mediated condition or being at a risk thereof.

The latter is achieved by comparing the expression level or amount of a pro-inflammatory gene/protein and/or of a chromatin remodelling gene/protein observed in a first sample from said subject to the expression level or amount of a pro-inflammatory gene/protein and/or of a chromatin remodelling gene/protein to a further sample from said subject as is described in more detail herein.

While p38 MAP kinase up-regulates genes involved in inflammatory responses such as pro-inflammatory genes in early stages after p38 MAP kinase has been induced by a stimulus such as LPS, for example, within 1, 2, 3 or preferably 4 hours, it down-regulates genes involved in inflammatory responses such as pro-inflammatory genes at a later stage, for example, after 5, 6, 7, 8, 9, 10, 11, 12, 13, 14, 15 or 16 or more hours after it has been induced.

Accordingly, in case of an inflammation such as an acute inflammation or a chronic inflammation it may be deleterious or even detrimental to administer a subject suffering a p38 mediated condition a p38 MAP kinase inhibitor, since in such subjects p38 MAP kinase may already have initiated an anti-inflammatory response as described herein. Thus, in some embodiments, the methods, uses and kits disclosed herein are in particular suitable for determining whether therapy with a p38 MAP kinase inhibitor is potentially beneficial or potentially contraindicated for a subject suffering from an inflammation (acute or chronic). Further details are described herein below.

Down-regulation of genes involved in inflammatory responses is assumed to be mediated by epigenetic silencing effected by chromatin remodelling genes which are surprisingly dependent on p38 MAP kinase. Chromatin remodelling genes are, for example, histone acetylases, histone deacetylases, histone methyltransferases or histone demethylases.

These findings are reflected in the present application. In fact, the present application teaches that p38 MAP kinase inhibitors which are currently proposed as novel anti-inflammatory agents might result in deleterious effects on the innate immune system and/or on innate immune-regulatory mechanisms, since inhibition of p38 MAP kinase at a false point of time during therapy with a p38 MAP kinase inhibitor might reverse an anti-inflammatory stage effected by epigenetic silencing of pro-inflammatory genes, which, in turn, would re-induce an inflammatory stage in a subject who has suffered from a p38-mediated condition such as inflammation or is still suffering thereof (see FIG. 10). Accordingly, the state of health of the subject, rather than being improved, might even be worsened.

The tremendous interest in p38 MAP kinase as a therapeutic for inflammatory diseases stems from an ever-growing body of data demonstrating the importance of the p38 pathway in the cellular response to inflammatory stimuli and the very broad efficacy of p38 MAP kinas inhibitors in preclinical models of disease. Indeed, in p38-β−/− mice it has recently been demonstrated that the LPS-induced cytokine response is normal, suggesting that p38 is the major target for p38 MAP kinase inhibitors (Geadmore et al. in Mol. Cell Biol. 25:10454-10464). Even more, the efficacy of a p38 MAP kinase inhibitor has been demonstrated in humans; see Branger et al. in J Immunol. 2002 Apr. 15; 168(8):4070-4077.

The innate immune system comprises the cells and mechanisms that defend the host from infection by other organisms, in a non-specific manner. This means that the cells of the innate system recognize, and respond to, pathogens in a generic way, but unlike the adaptive immune system, it does not confer long-lasting or protective immunity to the host. Innate immune systems provide immediate defense against infection.

In some cases, subjects who suffer from a p38-mediated condition may be identified in accordance with the teaching of the present application as not being eligible to a treatment with a p38 MAP kinase inhibitor as will be described herein.

Other subjects in accordance with the teaching of the present application may be identified as being eligible to a treatment with a p38 MAP kinase inhibitor as will also be described herein.

And also, the present application provides guidance as to whether or not a subject who suffers from a p38-mediated condition and who is already treated with a p38 MAP kinase inhibitor should be further treated with a p38 MAP kinase inhibitor.

In essence, the present application provides means and methods which allow a more targeted and/or tailored therapy with a p38 MAP kinase inhibitor.

Specifically, means and methods are provided which allow the skilled person to decide whether a treatment of a p38-mediated condition with a p38 MAP kinase inhibitor may potentially be beneficial or potentially contraindicated for a subject who suffers from a p38-mediated condition or is at a risk thereof.

Before the present invention is described in detail, it is to be understood that this invention is not limited to the particular methodology, protocols, and reagents etc. described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It must be noted that as used herein and in the appended claims, the singular forms “a”, “an”, and “the”, include plural referents unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods or uses described herein.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

In view of the findings of the present inventor, it is highly desirable to determine whether or not p38 MAP kinase may already have mediated an anti-inflammatory stage in a subject, preferably in immune cells of a subject such as T cells, B cells, NK cells, macrophages or monocytes, more preferably in monocytes of a subject.

The term “anti-inflammatory stage” means that in cells, preferably cells of the immune system such as T cells, B cells, NK cells, macrophages or monocytes genes involved in an inflammatory response are down-regulated and/or that these cells are no longer involved in an inflammatory response.

Preferably, these genes are down-regulated to about 10, 20, 30, 40, 50, 60, 70, 80, 90 or even 100% when compared to their induced state, i.e., during an inflammatory response. In the context of the present invention, an anti-inflammatory stage is, without being bound by theory, mediated, inter alia, by p38 MAP kinase via epigenetic silencing mechanisms as described herein.

In accordance with the teaching of the present invention, in a subject who suffers from a p38-mediated condition or is at risk of suffering from a p38-mediated condition, the presence of at least one chromatin remodelling gene described herein and/or of at least one pro-inflammatory gene described herein should be measured by the means and methods described herein.

If at least one chromatin remodelling gene is underrepresented in a sample obtained from a subject, who is suffering for a p38-mediated condition or at a risk suffering thereof, in comparison to a sample obtained from a subject who is not suffering from a p38-mediated condition or not at a risk suffering thereof and/or if at least one pro-inflammatory gene is overrepresented in a sample obtained from a subject, who is suffering from a p38-mediated condition or at a risk suffering thereof in comparison to a sample obtained from a subject who is not suffering from a p38-mediated condition or not at a risk suffering thereof, then it may be beneficial to treat such a subject suffering from a p38-mediated condition or being at a risk suffering thereof with a p38 MAP kinase inhibitor.

This is so, because the present inventor has observed that p38 MAP kinase mediates, apart from its role in inducing inflammatory responses including induction and maintenance of expression of genes involved in an inflammatory response such as pro-inflammatory genes, epigenetic silencing.

Epigenetic silencing down-regulates gene expression due to chromatin modification such as deacetylation or methylation and/or demethylation of histones. Accordingly, without being bound by theory, it is assumed that p38 MAP kinase dependent epigenetic silencing down-regulates genes involved in inflammatory responses such as pro-inflammatory genes. As such p38 MAP kinase can on the one hand induce inflammatory responses and on the other hand down-regulate inflammatory responses by inducing epigenetic silencing.

Epigenetic silencing, as mentioned before, can then down-regulate genes involved in inflammatory responses. From the foregoing, it is apparent that it is crucial to measure in a subject who is suffering from a p38-mediated condition or at a risk suffering thereof whether or not at least one chromatin remodelling gene is underrepresented and/or at least one pro-inflammatory gene is overrepresented in a sample obtained from such a subject in comparison to a sample obtained from a subject who is not suffering from a p38-mediated condition or not being at a risk suffering thereof.

In fact, treating a subject suffering from a p38-mediated condition or being at a risk suffering thereof with a p38 MAP kinase inhibitor in which at least one chromatin remodelling gene is overrepresented and/or at least one pro-inflammatory gene is underrepresented in a sample from such a subject in comparison to a sample obtained from a subject not suffering from a p38-mediated condition or not being at a risk suffering thereof, may reverse the anti-inflammatory stage already established in the subject suffering from a p38-mediated condition or being at a risk suffering thereof.

Accordingly, instead of achieving a beneficial treatment for such a subject, the opposite may be effected. Indeed, inhibiting p38 MAP kinase may then abolish the function of p38 MAP kinase in its role of mediating epigenetic silencing. Hence, for example, pro-inflammatory genes which have been down-regulated due to p38 MAP kinase dependent epigenetic silencing may again be induced and, thus, a p38-mediated condition in a subject who is already suffering from a p38-mediated condition could become even more severe or deleterious.

Likewise, in a subject who is at a risk of a p38-mediated condition, a p38-mediated condition could be induced, if a p38 MAP kinase inhibitor is administered, even though at least one chromatin remodelling gene is overrepresented and/or at least one pro-inflammatory gene is underrepresented in a sample obtained from such a subject in comparison to a sample obtained from a subject who is not suffering from a p38-mediated condition or not being at a risk suffering thereof.

Once the skilled person has measured in a subject suffering from a p38-mediated condition or being at a risk suffering thereof the presence of at least one chromatin remodelling gene and/or of at least one pro-inflammatory gene in a sample obtained from such a subject and has observed that at least one chromatin remodelling system is underrepresented and/or at least one pro-inflammatory gene is overrepresented in comparison to a sample obtained from a subject who is not suffering from a p38-mediated condition or not being at a risk suffering thereof, the skilled person knows that such a subject suffering from a p38-mediated condition or being a risk suffering thereof, could be treated with a p38 MAP kinase inhibitor.

It is assumed that the treatment should continue and should be beneficial as long as at least one chromatin remodelling system will be underrepresented and/or at least one pro-inflammatory gene will be overrepresented in a sample from the subject treated with a p38 MAP kinase inhibitor in comparison to a sample from the same subject before treatment with a p38 MAP kinase inhibitor.

Accordingly, in connection with the methods of the present invention, it is foreseen that a sample of a subject who is suffering from a p38-mediated condition or being at a risk suffering thereof is to be obtained prior to the treatment with a p38 MAP kinase inhibitor, during the treatment and/or after the treatment. Preferably, it is obtained prior to the treatment.

As mentioned before, if the treatment of a subject suffering from a p38-mediated condition with a p38 MAP kinase inhibitor is continued, even though at least one chromatin remodelling system is already overrepresented and/or at least one pro-inflammatory gene is already underrepresented in a sample obtained from such a subject in comparison to a sample from such a subject obtained prior to the treatment with a p38 MAP kinase inhibitor, then the p38 MAP kinase could no longer mediate p38 MAP kinase dependent epigenetic silencing, which would in turn lead to the re-induction of pro-inflammatory genes.

In essence, the prolonged treatment of such a subject with a p38 MAP kinase inhibitor may then have adverse effects or even deleterious effects for such a subject, since p38 MAP kinase could re-initiate an inflammatory stage which is undesirable.

The methods of the present invention are also applicable to a subject who suffers from a p38-mediated condition and is already treated with a p38 MAP kinase inhibitor.

Specifically, if a subject who suffers from a p38-mediated condition is already treated with a p38 MAP kinase inhibitor, without being bound by theory, at least one chromatin remodelling system should be overrepresented and/or at least one pro-inflammatory gene should be underrepresented in a sample obtained from such a subject in comparison to a sample obtained from a subject who does not suffer from a p38-mediated condition.

If so, the treatment is assumed to be beneficial. Preferably, a sample from such a subject should be obtained prior to the treatment with a p38 MAP kinase inhibitor for the purpose of comparison with the sample obtained from such a subject during the treatment with a p38 MAP kinase inhibitor.

Yet, if in a subject who suffers from a p38-mediated condition and who is already treated with a p38 MAP kinase inhibitor, at least one chromatin remodelling system is underrepresented and/or at least one pro-inflammatory gene is overrepresented in a sample obtained from such a subject in comparison to a sample obtained from a subject suffering from a p38-mediated condition, the skilled person is in a position to decide whether or not the treatment should be discontinued, since the p38 MAP kinase inhibitor may have reversed the p38 MAP kinase dependent epigenetic silencing, thereby re-inducing an inflammatory response by inducing genes such as pro-inflammatory genes, which, however, is not desirable.

Alternatively, it could also be that the treatment with the p38 MAP kinase inhibitor is to be continued, since it may not yet have effected inhibition of p38 MAP kinase so that p38 MAP kinase can still induce an inflammatory response, thereby inducing, e.g., pro-inflammatory genes and has not yet induced chromatin remodelling genes so as to later down-regulate, e.g., pro-inflammatory genes by epigenetic silencing.

In sum, p38 MAP kinase can induce an inflammatory stage, thereby inducing pro-inflammatory genes. After having induced an inflammatory stage, p38 MAP kinase can also mediate epigenetic silencing by inducing chromatin remodelling genes. Epigenetic silencing can then, without being bound to by theory, down-regulate a p38 MAP kinase induced inflammatory response. Thus, it is important that this natural shut-down of an inflammatory response is in balance.

The present invention provides thus means and methods to monitor the effects of p38 MAP kinase both on inducing an inflammatory response and inducing an epigenetic silencing effect which in turn can down-regulate the inflammatory response. This is, in particular, important to control a potentially exceeding inflammatory response such as a cytokine storm, which can be deleterious to a subject suffering from a p38-mediated condition. Cytokines being present during a cytokine storm are TNF, IL-1, IL-6 and IL-10.

For example, inhibiting p38 MAP kinase by an inhibitor may be beneficial for a subject suffering from a p38-mediated condition when at least one chromatin remodelling gene is underrepresented and/or at least one pro-inflammatory gene is overrepresented in a sample obtained from such a subject in comparison to a sample obtained from a subject who is not suffering from a p38-mediated condition. However, inhibiting p38 MAP kinase after it has induced an anti-inflammatory stage may have potentially deleterious effects for a subject suffering from a p38-mediated condition for the reasons explained hereinabove. In fact, without being bound by theory, inhibiting p38 MAP kinase, after it has induced an anti-inflammatory stage, may overcome the p38 MAP kinase dependent epigenetic silencing, thereby again inducing, e.g., pro-inflammatory genes which again induce an inflammatory stage. Thus, it is important to carefully control and/or monitor the presence of at least one chromatin remodelling gene and/or at least one pro-inflammatory gene in a sample obtained from a subject suffering from a p38-mediated condition or being at a risk suffering thereof, preferably prior to the treatment with a p38 MAP kinase inhibitor, during the treatment and/or after the treatment in order to decide whether or not the treatment should be started or discontinued and/or re-started again.

Likewise, controlling and/or monitoring the presence of at least one chromatin remodelling gene and/or at least one pro-inflammatory gene in a sample obtained from a subject suffering from a p38-mediated condition and who is already treated with a p38 MAP kinase inhibitor in comparison to a sample from a subject not suffering from a p38-mediated condition is important to be in a position to decide whether the treatment is already beneficial, if at least one chromatin remodelling system is overrepresented and/or at least one pro-inflammatory gene is underrepresented in comparison to a sample, preferably obtained from the same subject prior to the treatment or, alternatively, in a sample obtained from a subject who does not suffer from a p38-mediated condition.

Otherwise, if at least one chromatin remodelling gene is underrepresented and/or at least one pro-inflammatory gene is overrepresented, the treatment with a p38 MAP kinase inhibitor in a subject who is already treated with a p38 MAP kinase inhibitor may not yet be beneficial or may already become deleterious, since the p38 MAP kinase mediated epigenetic silencing effect may be reversed so that p38 MAP kinase re-induces an inflammatory stage in such a subject.

In such a case and in other embodiments described herein, it is preferable to measure the activity of, e.g., histone acetylases, histone methyltransferases and/or histone demethylases. Accordingly, if the activity of, e.g., histone acetylases, histone methyltransferases and/or histone demethylases is increased it may be indicative that the treatment with a p38 MAP kinase inhibitor is contraindicated and should be discontinued.

The term “therapy” when used herein means that a subject in need of a treatment of p38-mediated condition is treated with a p38 MAP kinase inhibitor. Thus, a “therapy”, as used herein, is an approach for obtaining beneficial or desired clinical results. For purposes of this invention, (beneficial or desired) clinical results include, but are not limited to, one or more of the following: alleviation of symptoms, diminishment of extent of a p38-mediated condition, stabilized (i.e., not worsening) state of a p38-mediated condition, preventing spread of a p38-mediated condition, preventing occurrence or recurrence of a p38-mediated condition, delay or slowing of a p38-mediated condition progression, amelioration of the a p38-mediated condition state, and remission (whether partial or total).

“Overrepresented” or “overrepresentation” and all its grammatical forms when used herein means that a gene or protein, in particular a pro-inflammatory gene or protein or a chromatin remodelling gene or protein is present more often, in a higher amount, is higher expressed etc. in a sample from a subject suffering from a p38-mediated condition in comparison to a reference sample. “More often” or in a “higher amount” means that the gene or protein is at least 10, 20, 30, 40, 50, 60, 70, 80, 90, 100, 110, 120, 130, 140, 150, 160, 170, 180, 190, 200, 250, 300, 350, 400, 450, 500-fold more often (%) or in a higher amount present in the sample from a subject suffering from a p38-mediated condition in comparison to a reference sample.

“Underrepresented” or “underrepresentation” and all its grammatical forms when used herein means that a gene or protein, in particular a pro-inflammatory gene or protein or a chromatin remodelling gene or protein is present less often, in a lower amount etc. in a sample from a subject suffering from a p38-mediated condition in comparison to a reference sample. “Less often” or in a “lower amount” means that the gene or protein is at least 10, 20, 30, 40, 50, 60, 70, 80, 90, 100, 110, 120, 130, 140, 150, 160, 170, 180, 190, 200, 250, 300, 350, 400, 450, 500-fold less often (%) or in a lower amount present in the sample from a subject suffering from a p38-mediated condition in comparison to a reference sample. “Less often” or “lower amount” also includes that a pro-inflammatory or chromatin remodelling gene or protein is present as often or in an equal amount in a sample from a subject suffering from a p38-mediated condition in comparison to a reference sample.

A “reference sample” is preferably a sample obtained from a subject not suffering from a p38-mediated condition, also sometimes called healthy subject herein. However, a reference sample also relates to a further, for example, second, third, fourth, fifth, etc. sample from a subject suffering from a p38-mediated condition or being at a risk of suffering thereof.

Accordingly, it is an embodiment of the present invention that all methods, uses and kits disclosed herein use as a reference sample a further, i.e., second, third, fourth, fifth, etc. sample from a subject suffering from a p38-mediated condition, wherein said subject is the same from which a first sample has been obtained. This allows monitoring within one and the same subject over-or underrepresentation of a pro-inflammatory gene or protein and/or of a chromatin remodelling gene or protein. Said further sample is preferably obtained about 1, 2, 3, 4, 5, 6, 7, 8, 9, 10, 11, 12, 13, 14, 15, 16, 17, 18, 19, 20, 21, 22, 23, 24, 28, 32, 36, 40, 44, 48, 52, 56, 60, 64, 68, 72, 76, 80, 84, 88, 92, 96, 100, 1004, 108, 112, 116, 120 hours after said first sample has been obtained, with 6, 12, 18 and 24 hours preferred.

Accordingly, it can then be determined as to whether a pro-inflammatory gene or protein is overrepresented or underrepresented, respectively, and/or as to whether a chromatin remodelling gene or protein is overrepresented or underrepresented. The skilled practitioner, in accordance with the teaching of the present invention, can then determine as to whether therapy with a p38 MAP kinase inhibitor may be beneficial or contraindicated and/or as to whether an already ongoing therapy may be continued or discontinued.

The term “p38 MAP kinase” or “p38” or “p38 MAPK” when used herein includes the four isoforms of p38 MAP kinase, i.e., p38-α(MAPK14), -β(MAPK11), -γ (MAPK12 or ERK6) and -δ(MAPK13 or SAPK4) as well as any further isoform(s) of p38 such as p38-32.

Kumar, S., et al., Biochem Biophys Res Comm (1997) 235:533-538 and Stein, B., et al., J Biol Chem (1997) 272:19509-19517 reported a second isoform of p38-β, -38-β2, containing 364 amino acids with 73% identity to p38-α. All of these reports show evidence that p38-β is activated by proinflammatory cytokines and environmental stress, although the second reported p38-β isoform, p38-β2, appears to be preferentially expressed in the central nervous system (CNS), heart and skeletal muscle, compared to the more ubiquitous tissue expression of p38-α. Furthermore, activated transcription factor-2 (ATF-2) was observed to be a better substrate for p38-β2 than for p38-α, thus suggesting that separate mechanisms of action may be associated with these forms. The physiological role of p38-β1 has been called into question by the latter two reports, since it cannot be found in human tissue and does not exhibit appreciable kinase activity with the substrates of p38-α.

The identification of p38-γ was reported by Li, Z., et al., Biochem Biophys Res Comm (1996) 228:334-340 and of p38-δ by Wang, X., et al., J Biol Chem (1997) 272:23668-23674 and by Kumar, S., et al., Biochem Biophys Res Comm (1997) 235:533-538. The data suggest that these two p38 isoforms (γ and δ) represent a unique subset of the MAPK family based on their tissue expression patterns, substrate utilization, response to direct and indirect stimuli, and susceptibility to kinase inhibitors.

p38-α and -β are closely related, but diverge from γ and δ, which are more closely related to each other. These protein kinases are activated by phosphorylation by upstream kinases MKK3, MKK6, and possibly by autophosphorylation. p38-α has been extensively studied and has been shown to play a causal role in inflammatory responses and in stress responses including apoptosis. p38-β has been less extensively studied. Nevertheless, these isoforms have distinct temporal and spatial patterns of expression, suggesting unique roles.

p38-α is the predominant isoform in leukocytes, epithelial cells, smooth muscle cells, whereas p38-δ has been observed to be more highly expressed in marophages and p38-γ in skeletal muscle.

Preferably, when used herein at least one, two, three or all four isoforms of p38 MAP kinase as well as any sub-form of an isoform is meant. More preferably, p38-α(MAPK14), -β(MAPK11), -γ (MAPK12 or ERK6) are meant which can be inhibited by a p38 MAP kinase inhibitor, preferably by using SB202190.

A “p38 MAP kinase inhibitor” is well known in the art. The terms “p38 inhibitor,” “p38 kinase inhibitor,” and “p38 MAP kinase inhibitor” are used interchangeably herein. In the context of the present invention a p38 MAP kinase inhibitor inhibits p38 MAP kinase. Accordingly, the p38 MAP kinase inhibitor is believed to inhibit the activity of p38 MAP kinase. Key activities are, for example, cytokine and chemokine expression, cell survival, TGF-β signalling and neutrophil function (e.g. superoxide burst). For example, a typical p38 MAP kinase inhibitor is believed to reduce levels of pro-inflammatory cytokines and chemokines and/or to reduce cellular infiltration to sites of inflammation, thereby reducing local damage.

Preferably, the p38 MAP kinase inhibitor inhibits one of the isoforms of p38 MAP kinase, preferably one of the four isoforms (α, β, γ or δ) of p38 MAP kinase with the α-isoform being preferred, more preferably it inhibits any combination of two isoforms of p38 MAP kinase, with the combination of p38-α/β preferred, even more preferably it inhibits any combination of three isoforms of p38 MAP kinase and most preferably, it inhibits all isoforms or the α, β, γ and δ isoform of p38 MAP kinase. In some embodiments, the p38 MAP kinase inhibitor inhibits the isoform of p38 that is involved in inflammatory diseases, autoimmune diseases, destructive bone disorders, proliferative disorders, infectious diseases, viral diseases or neurodegenerative diseases. It is reported that the α-isoform of p38 MAP kinase is involved in inflammation, proliferation, differentiation and apoptosis, whereas the biological functions of p38 β, p38 δ and p38 γ are not yet understood completely. Accordingly, it is preferred herein that the p38 MAP kinase inhibitor inhibits the α-isoform.

A p38 MAP kinase inhibitor can be a small molecule, large molecule, peptide, oligonucleotide, and the like. The p38 MAP kinase inhibitor may be a protein or fragment thereof or a nucleic acid molecule.

The determination of whether or not a compound is a p38 kinase inhibitor is within the skill of one of ordinary skill in the art. An example of how one would determine if a compound is a p38 kinase inhibitor would be to isolate the p38 kinase protein. The protein can be isolated from cells where the p38 kinase is naturally expressed or where it has been overexpressed by means of transfection of an oligonucleotide or infection with a virus that directs the expression of the p38 MAP kinase protein. Additionally, p38 can also be expressed recombinantly. Upon isolating the protein a person of ordinary skill in the art can measure the activity of the kinase in the presence or absence of a potential p38 kinase inhibitor. If the kinase activity is less in the presence than in the absence of an alleged inhibitor, that inhibitor is a p38 kinase inhibitor.

There are many examples of p38 inhibitors in the art. U.S. Pat. Nos. 5,965,583, 6,040,320, 6,147,096, 6,214,830, 6,469,174, 6,521,655 disclose compounds that are p38 inhibitors. U.S. Pat. Nos. 6,410,540, 6,476,031 and 6,448,257 also disclose compounds that are p38 inhibitors. Similarly, U.S. Pat. Nos. 6,410,540, 6,479,507 and 6,509,361 disclose compounds that are asserted to be p38 inhibitors. U.S. Published Application Nos. 20020198214 and 20020132843 disclose compounds that are said to be p38 inhibitors. Another p38 MAP kinase inhibitor is BIRB 796 BS (1-(5-tert-butyl-2-p-tolyl-2H-pyrazol-3-yl)-3-[4-(2-morpholin-4-yl-ethoxy)-naphthalen-1-yl]-urea, Doramapimod)); see Branger (2002), J. Immunol. 168:4070-4077 or US 6,319,921 for further p38 MAP kinase inhibitors.

Other p38 MAP kinase inhibitors are AMG 548 (Amgen), BIRB 796 (Boehringer Ingelheim), VX-702 and VX-745 (Vertex/Kissei), SCIO 469, SCIO 323 (Scios Inc.), SB 681323, SB-681323 and SB 242235 (GlaxoSmithKline), PH-797804 (Pfizer), TAK-715 (Takeda) and Org-48762-0 (Organon NV); see, for example, Lee and Dominguez in Curr Med Chem. 2005; 12(25):2979-2994, Dominguez in Curr Opin Drug Discov Devel. 2005 July; 8(4):421-430 and Mayer and Callahan in Drug Discovery Today: Therapeutic strategies 2006 Vol. 3, No. 1: 49-54.

According to the present invention, the inhibitor may exhibit its regulatory effect upstream or downstream of p38 MAP kinase or on p38 MAP kinase directly, with the latter mode of action being preferred. Examples of inhibitor regulated p38 MAP kinase activity include those where the inhibitor may decrease transcription and/or translation of p38 MAP kinase, may decrease or inhibit post-translational modification and/or cellular trafficking of p38 MAP kinase, or may shorten the half-life of p38 MAP kinase. The inhibitor may also reversibly or irreversibly bind p38 MAP kinase, inhibit its activation, inactivate its enzymatic activity, or otherwise interfere with its interaction with downstream substrates.

If acting on p38 MAP kinase directly, the inhibitor should exhibit an 1050 value of about 5 μM or less, preferably 500 nm or less, more preferably 100 nm or less. In a related embodiment, the inhibitor should exhibit an IC50 value relative to the p38-α isoform that is preferably at least ten fold less than that observed when the same inhibitor is tested against other p38 MAP kinase isoforms in the same or comparable assay. It should be noted that IC50 values are assay dependent and may change from determination to determination. It is more important to look at relative relationships of compounds' IC50 values rather than the exact values themselves.

The following assays can be used to determine relative IC50 values for p38 inhibitors. IC50 is the relative concentration of an inhibitor, which in the presence of the target kinase, causes a 50% decrease in kinase activity as compared to a control where the inhibitor is not present.

In an exemplary assay, compounds to be tested are solubilized in a suitable solvent, for example, DMSO or the like and diluted into water to the desired concentrations. The p38 kinase is diluted to 10 μg/ml into a buffer containing 20 mM MOPS, pH 7.0, 25 mM beta-glycerol phosphate, 2 mg/ml gelatin, 0.5 mM EGTA, and 4 mM DTT.

A reaction is carried out by mixing 20 μl test compound with 10 μl of a substrate cocktail containing 500 μg/ml peptide substrate and 0.2 mM ATP (+200 μCi/ml gamma-32P-ATP) in a 4× assay buffer. The reaction is initiated by the addition of 10 μl of p38 kinase. Final assay conditions are 25 mM MOPS, pH 7.0, 26.25 mM beta-glycerol phosphate, 80 mM KCl, 22 mM MgCl2, 3 mM MgSO4, 1 mg/ml gelatin, 0.625 mM EGTA, 1 mM DTT, 125 μg/ml peptide substrate, 50 μM ATP, and 2.5 μg/ml enzyme. After a 40 minute incubation at room temperature, the reaction is stopped by the addition of 10 μl per reaction of 0.25 M phosphoric acid.

A portion of the reaction is spotted onto a disk of P81 phosphocellulose paper, the filters are dried for 2 minutes and then washed 4× in 75 mM H₃PO₄. The filters are rinsed briefly in 95% ethanol, dried, then placed in scintillation vials with liquid scintillation cocktail.

Alternatively, the substrate, previously biotinylated, and the resulting reactions are spotted on SAM2™ streptavidin filter squares (Promega). The filters are washed 4× in 2M NaCl, 4× in 2M NaCl with 1% phosphoric acid, 2× in water, and briefly in 95% ethanol. The filter squares are dried and placed in scintillation vials with liquid scintillation cocktail.

Counts incorporated are determined on a scintillation counter. Relative enzyme activity is calculated by subtracting background counts (counts measured in the absence of enzyme) from each result, and comparing the resulting counts to those obtained in the absence of inhibitor. IC50 values are determined with curve-fitting plots available with common software packages.

In an alternative assay for measuring in vitro p38 kinase activity, human recombinant p38 (for example, the alpha or beta isoform) is mixed with the inhibitor at the desired concentration, and with DMSO kept at 1% in the final reaction. Myelin basic protein (MBP) and ATP are added and the reaction is incubated for 60 min at 25° C. The incubation conditions include 10 ug/ml MBP, 10 uM ATP, 50 mM HEPES, 20 mM MgCl₂, 0.2 mM Na VO₄, and 1 mM DTT, at pH 7.4. The degree of phosphorylation of the MBP is determined by ELISA quantitation of phospho-MBP.

As referenced above, IC50 is the concentration of compound which inhibits the enzyme to 50% of the activity as measured in the absence of an inhibitor.

IC50 values are calculated using the concentration of inhibitor that causes a 50% decrease as compared to a control. IC50 values are assay dependent and will vary from measurement to measurement. As such, IC50 values are relative values. The values assigned to a particular inhibitor are to be compared generally rather than on an absolute basis.

Samples or assays comprising MAP kinase that are treated with a potential activator, inhibitor, or modulator are compared to control samples without the inhibitor, activator, or modulator to examine the extent of inhibition. Control samples (untreated with inhibitors) can be assigned a relative MAP kinase activity value of 100%. Inhibition of MAP kinase is achieved when the MAP kinase activity value relative to the control is about 80%, optionally 50% or 25-0%. Activation of MAP kinase is achieved when the MAP kinase activity value relative to the control is 110%, optionally 150%, optionally 200-500%, or 1000-3000% higher. Exemplary MAP kinase binding activity assays of the present invention are: a MAP kinase ligand blot assay (Aymerich et al., Invest Opthalmol Vis Sci. 42:3287-93, 2001); a MAP kinase affinity column chromatography assay (Alberdi et al., J Biol Chem. 274:31605-12, 1999) and a MAP kinase ligand binding assay (Alberdi et al., J Biol Chem. 274:31605-12, 1999). Each incorporated by reference in their entirety.

The four isoforms of the p38 MAP kinase share a high level of sequence homology. The alpha and beta isoforms of the p38 MAP kinase are closely related while the gamma and delta isoforms are more divergent. Given the high degree of structural similarity, it is not surprising that certain compounds with the ability to inhibit one p38 MAP kinase isoform can often inhibit other isoforms of the MAP kinase. Accordingly, in some embodiments, an inhibitor of p38 MAP kinase that is specific for the α-isoform of the kinase possesses at least three categories of structural features that are theorized to permit isoform specific inhibition.

Selective binding of a candidate p38 MAP kinase inhibitor can be determined by a variety of methods. The genes for the various isoforms of p38 MAP kinase are known in the art. One of ordinary skill in the art could readily clone and express the various isoforms of the kinase, purify them, and then perform binding studies with candidate compounds to determine isoform binding characteristics. This series of experiments was performed for the α-isoform of p38 MAP kinase and provided in U.S. Pat. No. 6,617,324 B1.

Another kinase selectivity assay is described in Mihara (2008), Br. J. Pharmacol. 154(1):153-164.

In some embodiments herein, a p38 MAP kinase inhibitor inhibits an isoform of p38 MAP kinase, preferably one of the four isoforms (α, β, γ, δ) of p38 MAP kinase, with the α-isoform being preferred, more preferably it inhibits any combination of two isoforms of p38 MAP kinase, with the α- and β-isoform being preferred, even more preferably it inhibits any combination of three isoforms of p38 MAP kinase, e.g., p38-α(MAPK14), -β(MAPK11), -γ (MAPK12 or ERK6). Alternatively, but also preferred, it inhibits all four isoforms of p38 MAP kinase.

In the context of the present invention the term “subject” means an individual in need of a treatment of a p38-mediated condition. Preferably, the subject is a patient suffering from a p38-mediated condition or being at a risk thereof. Preferably, the patient is a mammal. Mammals include, but are not limited to, farm animals, sport animals, pets, primates, mice and rats. Preferably, a mammal is as a human, dog, cat, cow, pig, mouse, rat etc., particularly preferred, it is a human.

The term “potentially beneficial” when used in the context of the present application means that treating a subject with a p38 MAP kinase inhibitor is likely to ameliorate or improve one or more of the following aspects: alleviation of symptoms, diminishment of extent of a p38-mediated condition, stabilized (i.e., not worsening) state of a p38-mediated condition, preventing spread of a p38-mediated condition, preventing occurrence or recurrence of a p38-mediated condition, delay or slowing of a p38-mediated condition progression, amelioration of the a p38-mediated condition state, and remission (whether partial or total).

Accordingly, said term includes that one or more of the aforementioned aspects are ameliorated or improved to about 10, 20, 30, 40, 50, 60, 70, 80, 90 or even 100% in comparison to a subject suffering from a p38-mediated condition.

The term “potentially contraindicative” as used herein means that treating a subject with a p38 MAP kinase inhibitor does likely not ameliorate or improve one or more of the following aspects: alleviation of symptoms, diminishment of extent of a p38-mediated condition, stabilized (i.e., not worsening) state of a p38-mediated condition, preventing spread of a p38-mediated condition, preventing occurrence or recurrence of a p38-mediated condition, delay or slowing of a p 3 8-mediated condition progression, amelioration of the a p38-mediated condition state, and remission (whether partial or total).

Accordingly, said term includes that that one or more of the aforementioned aspects may become worsened or deteriorated. Thus, said term also includes that one or more of the aforementioned aspects are worsened or deteriorated to about 10, 20, 30, 40, 50, 60, 70, 80, 90 or even 100% in comparison to a subject not suffering from a p38-mediated condition.

In accordance with the present invention by the term “sample” is intended any biological sample obtained from an individual, cell line, tissue culture, or other source containing polynucleotides or polypeptides or portions thereof. As indicated, biological samples include body fluids (such as blood, sera, plasma, urine, synovial fluid and spinal fluid) and cells or tissue sources found to express the polynucleotides.

Preferably, the sample is obtained from peripheral blood mononuclear cells (PBMC) and contains immune cells such as T cells, B cells, NK cells, macrophages, monocytes etc. More preferably, the sample contains monocytes.

Methods for obtaining tissue biopsies and body fluids from mammals are well known in the art. A biological sample which includes genomic DNA, mRNA or proteins is preferred as a source.

When used herein a “pro-inflammatory gene” is a gene which is in particular expressed in an inflammatory response. Examples are cytokine genes encoding CCL-20, IL-6, CXCL-2, CCL-2, CXCL-3,CXCL-6, CD80, TNF, IL-1α, IL-1β, CCL5, IL-1, IL-6, IL-8, IL-17, IL-18, IL-23, interferon γ, platelet-activating factor (PAF), macrophage migration inhibitory factor (MIF), or high mobility group protein 1 (HMG-1). Another example of a pro-inflammatory gene is Dusp1 or Myc.

As used herein, the term “cytokine” refers to any secreted polypeptide that affects the functions of cells and is a molecule which modulates interactions between cells in the inflammatory response. A cytokine includes, but is not limited to, chemokines, monokines and lymphokines, regardless of which cells produce them.

In some embodiments of the present invention, the presence of at least one pro-inflammatory gene in a sample obtained from a subject who suffers from a p38-mediated condition or is at a risk thereof or who suffers from a p38-mediated condition and who is already treated with a p38 MAP kinase inhibitor is measured. Accordingly, for this measurement at least one pro-inflammatory gene is selected from the group consisting of CCL-20, IL-6, CXCL-2, CCL-2, CXCL-3,CXCL-6, CD80, TNF, IL-1α, IL-1β, CCL5, IL-1, IL-6, IL-8, IL-17, IL-18, IL-23, interferon γ, platelet-activating factor (PAF), macrophage migration inhibitory factor (MIF), high mobility group protein 1 (HMG-1). Alternatively, at least 2, 3, 4, 5, 6, 7, 8 , 9, 10, 11, 12, 13, 14, 15, 16, 17, 18, 19, 20, 25, 30, 35, 40, 45, 50 or more genes are selected for a measurement.

As a further alternative or addition to the aforementioned pro-inflammatory genes, one or more genes from GO:0006954 (inflammatory response), GO:0006935 (chemotaxis), GO:0008009 (chemokine activity), GO:0006916 (anti-apoptosis), GO: 0007267 (cell-cell signalling), GO:0004872 (receptor activity) and/or GO:0006955 (immune response) can be chosen. GO-numbers are available at www.geneontology.org; see FIG. 2, left panels “up-regulated”.

The term “chromatin remodelling gene” when used herein includes genes which are dependent on p38 MAP kinase. Their gene products are involved in methylation, demethylation, phosphorylation, ubiquitination sumoylation, ADP-ribosylation, deimination, or proline isomerization.

Preferably, chromatin remodelling genes of the present invention, i.e., the gene products of such genes are involved in epigenetic silencing, e.g., histondeacetylases, histonmethyltransferase or histone demethylases. Also included by the “chromatin remodeling genes” referred to herein are nucleosome assembly genes such as genes encoding histone proteins.

In some embodiments of the present invention, the presence of at least one chromatin remodelling gene in a sample obtained from a subject who suffers from a p38-mediated condition or is at a risk thereof or who suffers from a p38-mediated condition and who is already treated with a p38 MAP kinase inhibitor is measured. Accordingly, for this measurement at least one chromatin remodelling gene is selected from the group consisting of histondeacetylases, histonmethyltransferass, histone demethylases, histone genes, genes involved in methylation, demethylation, phosphorylation, ubiquitination sumoylation, ADP-ribosylation, deimination, or proline isomerization of histones. Alternatively, at least 2, 3, 4, 5, 6, 7, 8 , 9, 10, 11, 12, 13, 14, 15, 16, 17, 18, 19, 20, 25, 30, 35, 40, 45, 50 or more genes are selected for a measurement.

As an alternative or addition to the aforementioned chromatin remodelling genes, one or more genes from the following Table 1 can be chosen.

TABLE 1

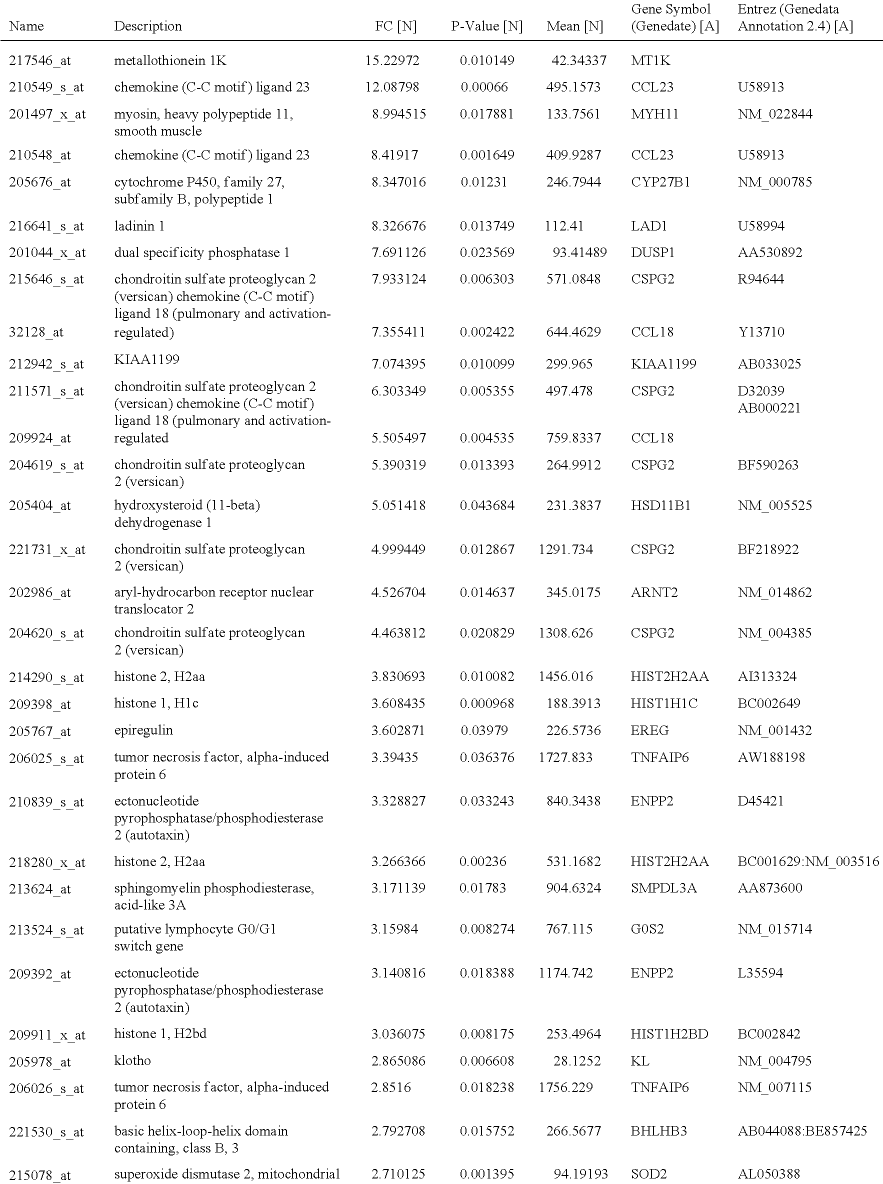

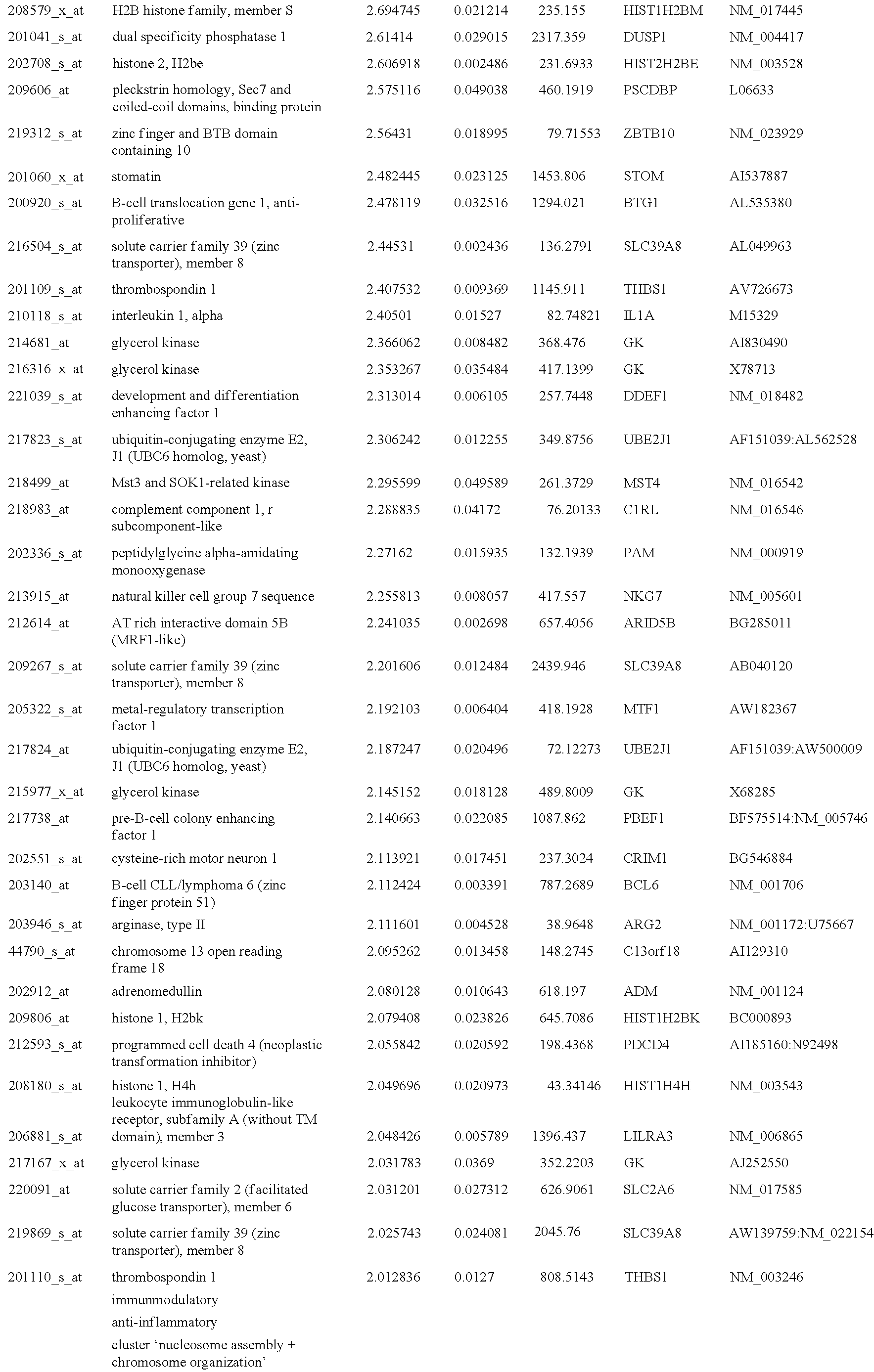

As a further alternative or addition to the aforementioned chromatin remodelling genes, one or more genes from GO:0005096 (GTPase activator activity), GO:0016491 (oxidoreductase activity), GO:0005975 (carbohydrate metabolism), GO:0005739 (mitochondrion), GO: 0005764 (lysosome), GO:0019884 (antigen presentation, exogenous antigen), GO:0045012 (MHC class II receptor activity), GO 0019886 (antigen processing, exogenous antigen via MHC class II), GO:0016787 (hydrolase activity)and/or GO:0006955 (immune response) can be chosen; see FIG. 2, right panels “down-regulated”. GO-numbers are available at www.geneontology.org.

Another alternative or addition to the aforementioned chromatin remodelling genes one or more genes from GO:0000786 (nucleosome), GO:0005694 (chromosome), GO:0006334 (nucleosome assembly), GO:0007001 (chromosome organization and biogenesis), GO:0007267 (cell-cell signaling), GO:0006954 (inflammatory response), GO:0003677 (DNA binding) and GO:0005576 (extracellular region) can be chosen; see FIG. 5. GO-numbers are available at www.geneontology.org.

In some embodiments, the presence of at least one chromatin remodelling gene and/or at least one pro-inflammatory gene is preferably measured by generating an expression profile from a subject who suffers from a p38-mediated condition or who is at a risk thereof.

In other embodiments, the presence of at least one chromatin remodelling gene and/or at least one pro-inflammatory gene is preferably measured by generating an expression profile from a subject who suffers from a p38-mediated condition and who is already treated with a p38 MAP kinase inhibitor.

In some embodiments, the presence of at least one chromatin remodelling gene and/or at least one pro-inflammatory gene is preferably measured by generating an expression profile from a subject who does not suffer from a p38-mediated condition or who is not at a risk thereof. Examples for generating expression profiles are given in Viemann et al. (2004), Blood 103:3365-3373 or Viemann et al. (2005), Biochem. Biophys. Acta 1746:73-84.

Such an expression profile is used as a reference expression profile. Thus, typically, the one or more reference expression profiles include a reference expression profile representing a disease-free subject, preferably a human, i.e., a subject who is not suffering from a p38-mediated condition or who is not at a risk suffering thereof.

An “expression profile” or “gene expression profile” is the measurement of the activity (the expression) of at least one gene, 10, 100 or thousands of genes at once, to create a global picture of cellular function. Typically, microarray technology is used which measures the relative activity of previously identified target genes. Tag-based techniques, like serial analysis of gene expression (SAGE, SuperSAGE) are also used for gene expression profiling. Microarrays are commercially available and more common. Deep sequencing is an emerging alternative to microarray gene profiling which is also envisaged to be applied in connection with the present invention.

Specifically, the gene expression profile measuring the presence of at least one chromatin remodelling gene and/or the presence of at least one pro-inflammatory gene may be compared to the reference expression profiles by, for example, a k-nearest neighbor analysis or a weighted voting algorithm. Typically, the expression profile represents known or determinable clinical outcomes of p38-mediated conditions.

In some embodiments, the gene expression profile from the subject who suffers from a p38-mediated condition or is at a risk thereof may be compared to at least two reference expression profiles, each of which represents a different clinical outcome. For example, each reference expression profile may represent a different clinical outcome selected from the group consisting of remission to less than 5, 10, 20, 30, 40, 50, 60, 70, 80, 90 and 100% in response to a p38 MAP kinase inhibitor treatment.

In some embodiments, the gene expression profile may be generated by using a microarray technology or tag-based techniques, like serial analysis of gene expression (SAGE, SuperSAGE). Microarrays are commercially available and more common. Deep sequencing is an emerging alternative to microarray gene profiling which is also envisaged to be applied in the means and methods of the present invention.

In some embodiments, the gene expression profile is generated when a subject is already treated with a p38 MAP kinase inhibitor.

In another embodiment, the one or more prognostic genes, which allow to determine whether a therapy with a p38 MAP kinase inhibitor may be potentially beneficial or contraindicated, which can be used in the context of the means and methods of the present invention include 5, 10, 15, 20, 25, 30, 40, 45 or more genes selected from Table 1, above, or Table 2 which follows.

TABLE 2

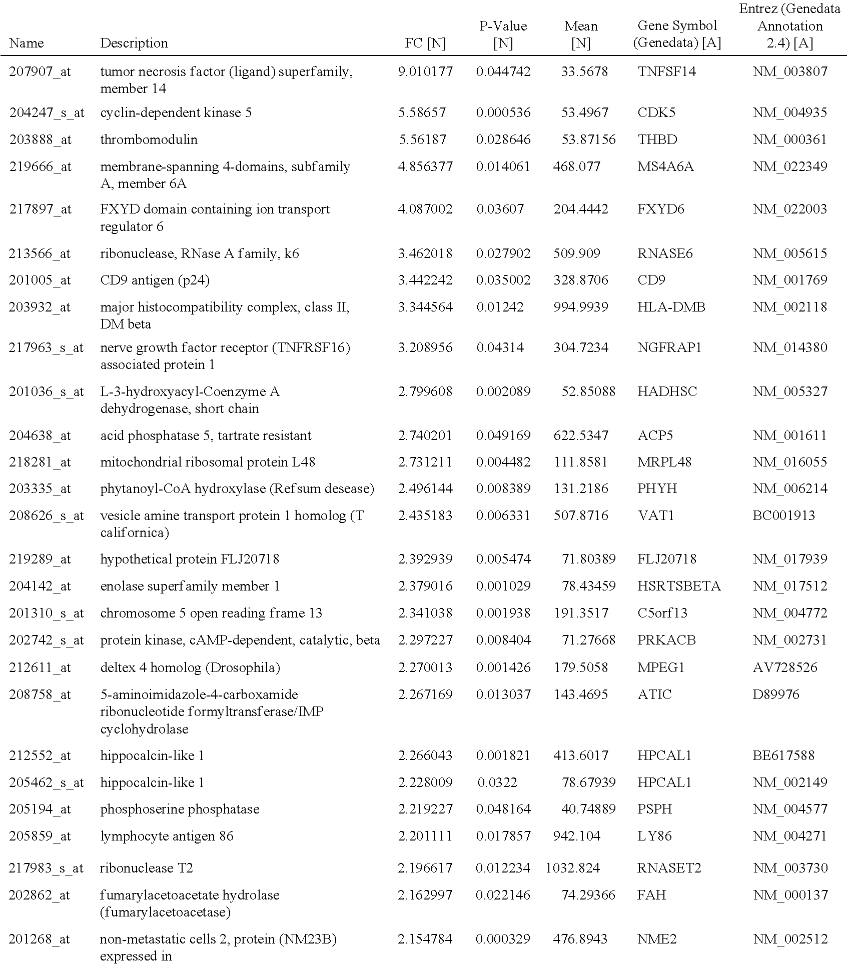

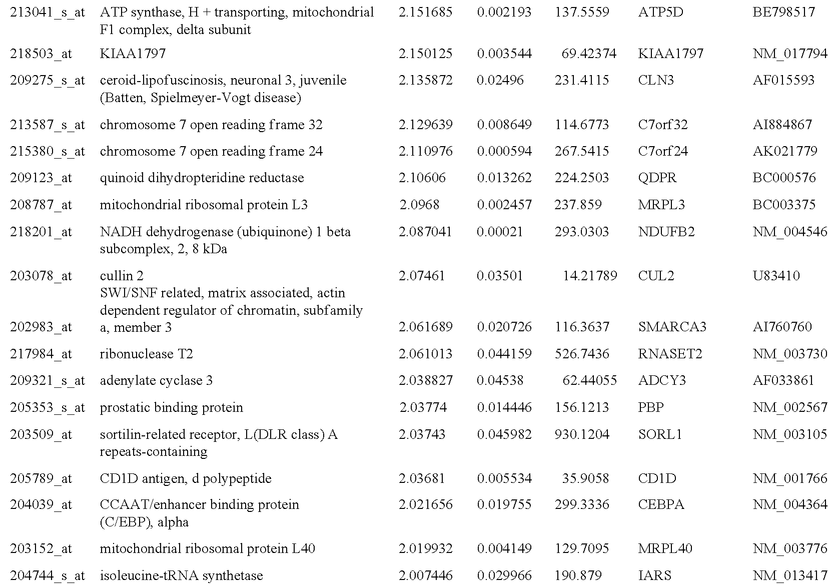

The relationship between the gene expression profile between a subject who suffers from a p38-mediated condition or who is at a risk thereof or a subject who suffers from a p38-mediated condition and who is already treated with a p38 MAP kinase inhibitor, respectively, and a reference expression profile can be evaluated by using global gene expression analyses. Methods suitable for this purpose include, but are not limited to, nucleic acid arrays (such as cDNA or oligonucleotide arrays), 2-dimensional SDS-polyacrylamide gel electrophoresis/mass spectrometry, and other high throughput nucleotide or polypeptide detection techniques.

Nucleic acid arrays allow for quantitative detection of the expression levels of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays.

The polynucleotides to be hybridized to a nucleic acid array can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats.

In the absolute hybridization format, polynucleotides derived from one sample, such as PBMCs from a subject, are hybridized to the probes on a nucleic acid array. Signals detected after the formation of hybridization complexes correlate to the polynucleotide levels in the sample. The nucleic acid array is then examined under conditions in which the emissions from the labels are detectable. In one embodiment, the fluorophores Cy3 or Cy5 (Amersham Pharmacia Biotech, Piscataway N.J.) are used as the labeling moieties for the differential hybridization format.

Signals gathered from a nucleic acid array can be analyzed using commercially available software, such as those provided by Affymetrix or Agilent Technologies. Controls, such as for scan sensitivity, probe labeling and cDNA/cRNA quantitation can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels of the genes are normalized across the samples such that the mean is zero and the standard deviation is one. In another embodiment, the expression data detected by nucleic acid arrays are subject to a variation filter which excludes genes showing minimal or insignificant variation across all samples.

The gene expression data collected from nucleic acid arrays can be correlated with the decision as to whether or not treatment with a p38 MAP kinase inhibitor is potentially beneficial or potentially contraindicated for a subject suffering from a p38-mediated condition using a variety of methods.

Methods suitable for this purpose include, but are not limited to, statistical methods (such as Spearman's rank correlation, Cox proportional hazard regression model, ANOVA/t test, or other rank tests) and class-based correlation metrics (such as nearest-neighbor analysis).

In one embodiment, subjects suffering from a p38-mediated condition or being at a risk thereof are divided into at least two classes based on their responses to a therapeutic treatment. The correlation between peripheral blood gene expression and the subject's response to a treatment with a p38 MAP kinase inhibitor is then analyzed by a supervised cluster or learning algorithm. Supervised algorithms suitable for this purpose include, but are not limited to, nearest-neighbor analysis, support vector machines, the SAM method, artificial neural networks, and SPLASH.

In another embodiment, subjects suffering from a p38-mediated condition or being at a risk thereof can be divided into at least two classes based on their peripheral blood gene expression profiles. Methods suitable for this purpose include unsupervised clustering algorithms, such as self-organized maps (SOMs), k-means, principal component analysis, and hierarchical clustering. A substantial number (e.g., at least 50%, 60%, 70%, 80%, 90%, or more) of subjects in one class may have a first clinical outcome, and a substantial number of patients in another class may have a second clinical outcome. Genes that are differentially expressed in the peripheral blood cells of one class of subjects relative to another class of subjects can be identified. These genes can also be used as prognostic markers for deciding whether or not therapy with a p38 MAP kinase inhibitor may be potentially beneficial or potentially contraindicated for a subject suffering from a p38-mediated condition or being at a risk thereof.

In yet another embodiment, subjects suffering from a p38-mediated condition or being at a risk thereof can be divided into three or more classes based on their clinical outcomes or peripheral blood gene expression profiles. Multi-class correlation metrics can be employed to identify genes that are differentially expressed in one class of subjects relative to another class. Exemplary multi-class correlation metrics include, but are not limited to, those employed by GeneCluster 2 software provided by MIT Center for Genome Research at Whitehead Institute (Cambridge, Mass.).

Other methods capable of determining the presence of at least one chromatin remodelling gene and/or at least one pro.-inflammatory gene include, but are not limited, RT-PCR, Northern Blot, in situ hybridization, and immunoassays such as ELISA, RIA or Western Blot. These genes are differentially expressed in, for example, peripheral blood cells {e.g., PBMCs) of subjects suffering from a p38-mediated condition or being at a risk thereof and subjects not suffering from a p38-mediated condition or not being at a risk thereof.

In many cases, the average peripheral blood expression level of each of these genes in one class of subjects is statistically different from that in another class of patients. For instance, the p-value under an appropriate statistical significance test (e.g., Student's t-test) for the observed difference can be no more than 0.05, 0.01, 0.005, 0.001, 0.0005, 0.0001, or less. In many other cases, each prognostic gene thus identified has at least 2-, 3-, A-, 5-, 10-, or 20-fold difference in the average PBMC expression level between one class of subjects and another class of subjects.

The peripheral blood samples which are preferably used in the present invention can be either whole blood samples, or samples comprising enriched PBMCs. In one example, the peripheral blood samples used for preparing the reference expression profile(s) comprise enriched or purified PBMCs, and the peripheral blood sample used for preparing the expression profile of the subject of interest, i.e., one which suffers from a p38-mediated condition or is at a risk thereof or is suffering from a p38-mediated condition and is already treated with a p38 MAP kinase inhibitor is a whole blood sample. In another example, all of the peripheral blood samples comprise enriched or purified PBMCs. In many cases, the peripheral blood samples are prepared from the subject of interest and reference subjects using the same or comparable procedures.

Other types of blood samples can also be employed in the present invention, and the gene expression profiles in these blood samples are statistically significantly correlated with the outcome of a subject who is treated with a p38 MAP kinase inhibitor.

The peripheral blood samples used in the present invention can be isolated from respective subjects at any stage of a p38-mediated condition with which they are affected. In many embodiments, clinical outcome is measured by subjects' response to a therapeutic treatment with a p38 MAP kinase inhibitor, and all of the blood samples used in outcome prediction are preferably isolated prior to the therapeutic treatment. The expression profiles derived from these blood samples are therefore baseline expression profiles for the therapeutic treatment.

Construction of the expression profiles typically involves detection of the expression level of each prognostic gene used in the outcome prediction. Numerous methods are available for this purpose. For instance, the expression level of a gene can be determined by measuring the level of the RNA transcript(s) of the gene. Suitable methods include, but are not limited to, quantitative RT-PCR, Northern Blot, in situ hybridization, slot-blotting, nuclease protection assay, and nucleic acid array (including bead array). The expression level of a gene can also be determined by measuring the level of the polypeptide(s) encoded by the gene. Suitable methods include, but are not limited to, immunoassays (such as ELISA, RIA, FACS, or Western blot), 2-dimensional gel electrophoresis, mass spectrometry, or protein arrays.

In one aspect, the expression level of a prognostic gene is determined by measuring the RNA transcript level of the gene in a peripheral blood sample. RNA can be isolated from the peripheral blood sample using a variety of methods. Exemplary methods include guanidine isothiocyanate/acidic phenol method, the TRIZOL® Reagent (Invitrogen), or the Micro-FastTrack™ 2.0 or FastTrack™ 2.0 mRNA Isolation Kits (Invitrogen). The isolated RNA can be either total RNA or mRNA. The isolated RNA can be amplified to cDNA or cRNA before subsequent detection or quantitation. The amplification can be either specific or non-specific. Suitable amplification methods include, but are not limited to, reverse transcriptase PCR (RT-PCR), isothermal amplification, ligase chain reaction, and Qbeta replicase.

In one embodiment, the amplification protocol employs reverse transcriptase.

The isolated mRNA can be reverse transcribed into cDNA using a reverse transcriptase, and a primer consisting of oligo (dT) and a sequence encoding the phage T7 promoter. The cDNA thus produced is single-stranded. The second strand of the cDNA is synthesized using a DNA polymerase, combined with an RNase to break up the DNA/RNA hybrid. After synthesis of the double-stranded cDNA, T7 RNA polymerase is added, and cRNA is then transcribed from the second strand of the doubled-stranded cDNA. The amplified cDNA or cRNA can be detected or quantitated by hybridization to labeled probes. The cDNA or cRNA can also be labeled during the amplification process and then detected or quantitated.

In another embodiment, quantitative RT-PCR (such as TagMan, ABI) is used for detecting or comparing the RNA transcript level of a prognostic gene of interest. Quantitative RT-PCR involves reverse transcription (RT) of RNA to cDNA followed by relative quantitative PCR (RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, in one embodiment, the sampling and quantifying of the amplified PCR products are carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs, While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In yet another embodiment, nucleic acid arrays (including bead arrays) are used for detecting or comparing the expression profiles of a prognostic gene described herein. The nucleic acid arrays can be commercial oligonucleotide or cDNA arrays. They can also be custom arrays comprising concentrated probes for the prognostic genes of the present invention, In many examples, at least 15%, 20%, 25%, 30%, 35%, 40%, 45%, 50%, or more of the total probes on a custom array of the present invention are probes for prognostic genes. These probes can hybridize under stringent or nucleic acid array hybridization conditions to the RNA transcrpts, or the complements thereof, of the corresponding prognostic genes.

As used herein, “stringent conditions” are at least as stringent as, for example, conditions G-L shown in Table 10. “Highly stringent conditions” are at least as stringent as conditions A-F shown in Table 10. Hybridization is cared out under the hybridization conditions (Hybridization Temperature and Buffer) for about four hours, followed by two 20-minute washes under the corresponding wash conditions (Wash Temp, and Buffer).

In one example, a nucleic acid array of the present invention includes at least 2, 5, 10, or more different probes. Each of these probes is capable of hybridizing under stringent or nucleic acid array hybridization conditions to a different respective prognostic gene of the present invention. Multiple probes for the same prognostic gene can be used on the same nucleic acid array. The probe density on the array can be in any range.

The probes for a prognostic gene of the present invention can be a nucleic acid probe, such as, DNA, RNA, PNA, or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the prognostic genes can be stably attached to discrete regions on a nucleic acid array. By “stably attached,” it means that a probe rnaintains its position relative to the attached discrete region during hybridization and signal detection. The position of each discrete region on the nucleic acid array can be either known or determinable.

In another embodiment, nuclease protection assays are used to quantitate RNA transcript levels in peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. Examples of suitable nuclease protection assays include the RNase protection assay provided by Ambion, Inc. (Austin, Tex.).

Hybridization probes or amplification primers for the prognostic genes of the present invention can be prepared by using any method known in the art. For prognostic genes whose genomic locations have not been determined or whose identities are solely based on EST or mRNA data, the probes/primers for these genes can be derived from the target sequences of the corresponding qualifiers, or the corresponding EST or mRNA sequences.

In one embodiment, the probes/primers for a prognostic gene significantly diverge from the sequences of other prognostic genes. This can be achieved by checking potential probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. The initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always>0) and N (penalty score for mismatching residues; always<0). The BLAST algorithm parameters W, T, and X determine the sensitivity and speed of the alignment. These parameters can be adjusted for different purposes, as appreciated by those skilled in the art.

In another embodiment, the probes for prognostic genes can be polypeptide in nature, such as, antibody probes.

The expression levels of the prognostic genes of the present invention, in particular pro-inflammatory and/or chromatin remodelling genes as described herein are thus determined by measuring the levels of polypeptides encoded by the prognostic genes. Methods suitable for this purpose include, but are not limited to, immunoassays such as ELISA, RIA, FACS, dot blot, Western Blot, immunohistochemistry, and antibody-based radioimaging. In addition, high-throughput protein sequencing, 2-dimensional SDS-polyacrylamide gel electrophoresis, mass spectrometry, or protein arrays can be used. Standard immunoassays for measuring the level of pro.inflammatory polypeptides encoded by the prognostic genes of the present invention, in particular cytokines, are known and available in the art.

In one embodiment, ELISAs are used for detecting the levels of the target proteins. In an exemplifying ELISA, antibodies capable of binding to the target proteins are immobilized onto selected surfaces exhibiting protein affinity, such as wells in a polystyrene or polyvinylchloride microtiter plate. Samples to be tested are then added to the wells. After binding and washing to remove non-specifically bound immunocomplexes, the bound antigen(s) can he detected. Detection can he achieved by the addition of a second antibody which is specific for the target proteins and is linked to a detectable label.

Detection can also be achieved by the addition of a second antibody, followed by the addition of a third antibody that has binding affinity for the second antibody, with the third antibody being linked to a detectable label. Before being added to the microtiter plate, cells in the samples can be lysed or extracted to separate the target proteins from potentially interfering substances.

In another exemplifying EUSA, the samples suspected of containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly, The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of theft labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation, These conditions may include, for example, diluting the antigens and antibodies with solutions such as BSA, bovine gamma globulin (BOG) and phosphate buffered saline (PBS)/Tween and incubating the antibodies and antigens at room temperature for about 1 to 4 hours or at 4° C overnight. Detection of the immunocomplex is facilitated by using a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation {e.g., incubation for 2 hours at room temperature in a PBS-containing solution such as PBS-Tween).

After incubation with the labeled antibody, and subsequent washing to remove unbound material, the amount of label can be quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for detecting polypeptide levels is RIA (radioimmunoassay). An exemplary RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this Standard curve, the concentration of the polypeptide in unknown samples can be determined. Protocols for conducting RIA are well known in the art.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used. Methods for preparing these antibodies are well known in the art. In one embodiment, the antibodies of the present invention can hind to the corresponding prognostic gene products or other desired antigens with binding affinities of at least 10⁴ M⁻¹, 10⁵ M⁻¹, 10⁶ M⁻¹, 10⁷ M⁻¹, or more.

The antibodies of the present invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The antibodies of the present invention can be used as probes to construct protein arrays for the detection of expression profiles of the prognostic genes. Methods for making protein arrays or biochips are well known in the art. In many embodiments, a substantial portion of probes on a protein array of the present invention are antibodies specific for the prognostic gene products. For instance, at least 10%, 20%, 30%, 40%, 50%, or more probes on the protein array can be antibodies specific for the prognostic gene products.

In yet another aspect, the expression levels of the prognostic genes are determined by measuring the biological functions or activities of these genes. Where a biological function or activity of a gene is known, suitable in vitro or in vivo assays can be developed to evaluate the function or activity. These assays can be subsequently used to assess the level of expression of the prognostic gene.

After the expression level of each prognostic gene is determined, numerous approaches can be employed to compare expression profiles. Comparison of the expression profile of a patient of interest to the reference expression profile(s) can be conducted manually or electronically. In one example, comparison is carried out by comparing each component in one expression profile to the corresponding component in a reference expression profile. The component can be the expression level of a prognostic gene, a ratio between the expression levels of two prognostic genes, or another measure capable of representing gene expression patterns. The expression level of a gene can have an absolute or a normalized or relative value. The difference between two corresponding components can be assessed by fold changes, absolute differences, or other suitable means.

In eukaryotes, the accessibility of large regions of DNA can depend on its chromatin structure which can be altered as a result of histone modifications. In general, the density of its packing is indicative of the frequency of transcription.

Chromatin is the complex of DNA and protein that makes up chromosomes. It is found inside the nuclei of eukaryotic cells. The major proteins involved in chromatin are histone proteins, although many other chromosomal proteins have prominent roles too. The functions of chromatin are to package DNA into a smaller volume to fit in the cell, to strengthen the DNA to allow mitosis and meiosis, and to serve as a mechanism to control expression and DNA replication. Changes in chromatin structure are affected by chemical modifications of histone proteins such as methylation (DNA and proteins) and acetylation (proteins), and by non-histone, DNA-binding proteins such as chromatin-bound enzymes, high mobility group (HMG) proteins, transcription factors, scaffold proteins, transition proteins or protamines.

Histones are composed of an octamer of the four core histones (H3, H4, H2A, H2B) around which 147 base pairs of DNA are wrapped. They are responsible for the amount of supercoiling of DNA, and these complexes can be temporarily modified by processes such as phosphorylation or more permanently modified by processes such as methylation. Such modifications are considered to be responsible for more or less permanent changes in gene expression levels and are effected by epigenetic silencing.

Epigenetic silencing is a general term describing epigenetic processes of gene regulation. The term epigenetic gene silencing is generally used to describe the “switching off” of a gene by a mechanism other than genetic modification. That is, a gene which would be expressed (turned on) under normal circumstances is switched off by machinery in the cell. Transcriptional gene silencing is the result of histone modifications, creating an environment of heterochromatin around a gene that makes it inaccessible to transcriptional machinery (RNA polymerase, transcription factors, etc.).

As mentioned, in the context of the present invention, chromatin remodelling genes are preferably dependent on p38 MAP kinase. More preferably, chromatin remodelling genes are genes whose gene products are involved in methylation, demethylation, phosphorylation, ubiquitination sumoylation, ADP-ribosylation, deimination, or proline isomerization of histones.

Preferably, chromatin remodelling genes of the present invention, i.e., the gene products of such genes are involved in epigenetic silencing, e.g., histone deacetylases, histone methyltransferases or histone demethylases.

Specifically, the present inventor has shown that inhibition of p38 MAP kinase by an inhibitor which is capable of inhibiting the p38 MAP kinase-α, -β, and -γ isoforms reverts an anti-inflammatory stage in immune cells, preferably in monocytes. p38 MAP kinase inhibitors are generally known in the art. An example for a p38 MAP kinase inhibitor which is capable of inhibiting the p38 MAP kinase-α, β, and -γ isoforms is SB202190 which is described in J Biol Chem 1997; 272(48):30122-30128.

Involvement of p38 MAP kinase in epigenetic silencing effects has been demonstrated by inhibition experiments; see FIG. 4. Specifically, late-stage LPS-induced gene expression changed significantly if long-term LPS-treated monocytes were treated with the p38 MAP kinase inhibitor SB 202190, while treating long-term LPS-induced monocytes with inhibitors of ERK kinase (PD98059) and PKC (GF109203X) did not significantly change gene expression.

However, the inhibition of p38 MAP kinase reversed the gene expression pattern and thus the activity of monocytes. In particular, chromatin remodelling and nucleosome assembly genes were observed to be down-regulated, while pro-inflammatory genes were again up-regulated.

This observation was confirmed by chromatin immunoprecipitation assays. Specifically, it was found that p38 MAP kinase inhibition induced acetylation and/or methylation on pro-inflammatory genes such as VCAM, IL-8, DUSP1, TNF or MYC.

Accordingly, in the alternative or in addition to measuring the presence of at least one pro-inflammatory gene and/or chromatin remodelling gene as described herein in the context of methods, uses and means, the acetylation and/or methylation status of one or more of the pro-inflammatory genes described herein and/or that of VCAM, IL-8, DUSP1, TNF and/or MYC can be measured and/or determined in order to determine whether therapy with a p38 MAP kinase inhibitor is potentially beneficial or potentially contraindicated for a subject suffering from a p38-mediated condition or being at a risk thereof.

The acetylation and/or methylation status can be measured by chromatin immunoprecipitation assays known in the art. Alternatively, the acetylation and/or methylation status can be measured by monitoring the activity of histone methyltransferases, histone demethylases, histone acetyltransferases and/or histone deacetylases.

Specifically, if the acetylation status, in particular of H3 and/or H4, and/or the methylation status, in particular of H3K4 and/or H3K49, of one or more of the pro-inflammatory genes described herein is increased in a subject who suffers from a p38-mediated condition or is at a risk thereof, then therapy with a p38 MAP kinase inhibitor can be potentially beneficial for the subject.

However, if the methylation status of H3K9 and/or H3K27 is increased, then a treatment with a p38 MAP kinase inhibitor may be potentially contraindicated.

Yet, if the acetylation status, in particular H3 and/or H4, and/or methylation status, in particular of H3K4 and/or H3K49, of one or more of the pro-inflammatory genes described herein is decreased in a subject who suffers from a p38-mediated condition or is at a risk thereof, then therapy with a p38 MAP kinase inhibitor can be potentially contraindicated for the subject.

However, if the methylation status of H3K9 and/or H3K27 is decreased, then a treatment with a p38 MAP kinase inhibitor may be potentially beneficial.

Histone deacetylases (HDAC) are a class of enzymes that remove acetyl groups from an ε-N-acetyl lysine amino acid on a histone. Its action is opposite to that of histone acetyltransferase.

Histone acetyltransferases (HAT) are enzymes that acetylate conserved lysine amino acids on histone proteins by transferring an acetyl group from acetyl CoA to lysine to form ε-N-acetyl lysine.

Histone acetylation is also an important process in transcription and is generally linked to transcriptional activation.

Histone acetyltransferase enzymes (HATs) such as CREB-binding protein also dissociate the DNA from the histone complex, allowing transcription to proceed. Often, DNA methylation and histone deacetylation work together in gene silencing. The combination of the two seems to be a signal for DNA to be packed more densely, lowering gene expression.

HDAC proteins are found in three groups, the first two groups belong to the classical HDACs and their activities are inhibited by trichostatin A (TSA) whereas the third group is a family of NAD+-dependent proteins not affected by TSA. Homologues to all three groups are found in yeast having the names reduced potassium dependency 3 (Rpd3)—corresponds to class 1, histone deacetylase 1 (hda1)—to class 2 and silent information regulator 2(Sir2)—class3.

In general, histone methylation effected by histone methyltransferases is associated with transcriptional repression.

Histone methylation is the modification of certain amino acids in a histone protein by the addition of one, two, or three methyl groups. This modification alters the properties of the nucleosome and affects its interactions with other proteins.

Histone methyltransferases (HMT) are enzymes, histone-lysine N-methyltransferase and histone-arginine N-methyltransferase, which catalyze the transfer of one to three methyl groups from the cofactor S-Adenosyl methionine to lysine and arginine residues of histone proteins.

Histone methylation serves in epigenetic gene regulation. Methylated histones bind DNA more tightly, which inhibits transcription.

However, methylation of some lysine and arginine residues of histones results in transcriptional activation. Examples include methylation of lysine 4 of histone 3 (H3K4), and arginine (R) residues on H3 and H4.

There are two families of histone demethylating enzymes. The first was Lysine Specific Demethylase 1 (LSD1) which is an flavin-dependent monoamine oxidase which can demethylate mono- and di-methylated lysines, specifically histone 3, lysines 4 and 9 (H3K4 and H3K9). This enzyme cannot demethylate tri-methylated lysines and for a short while it was thought that tri-methylated lysines may indeed be permanent modifications.

In addition, the Jumonji domain-containing (JmjC) histone demethylases were discovered which are able to demethylate mono-, di-, or tri-methylated lysines thereby disproving the theory that histone methylation is permanent once and for all.

“A p38-mediated condition” in accordance with the present invention means any disease or other deleterious condition in which p38 is known to play a role. This includes conditions which are known to be caused by conditions including TNF, 1α, IL-1β, IL-6, IL-8, IL-18, interferon γ, platelet-activating factor (PAF), macrophage migration inhibitory factor (MIF) overproduction. In other words, these conditions are cytokine-mediated disease since in particular p38alpha kinase is involved in the biosynthesis of cytokines such as tumor necrosis factor-alpha and interleukin-1 beta at the translational and transcriptional level. MAPK p38alpha thus represents a point of convergence for multiple signaling processes that are activated during inflammation, making it a key potential target for the modulation of cytokine production. p38-mediated condition include, without limitation, inflammatory diseases, autoimmune diseases, destructive bone disorders, proliferative disorders, infectious diseases, viral diseases, and neurodegenerative diseases.

An inflammatory disease includes “inflammation” or an “inflammatory response” which refers to an innate immune response that occurs when tissues are injured by, for example, bacteria, trauma, toxins, heat, or any other cause. The damaged tissue releases compounds including histamine, bradykinin, and serotonin. Inflammation refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

Accordingly, a p38-mediated condition as referred herein also includes inflammation or an inflammatory response from which a subject may have or which may occur in a subject.

Inflammatory diseases are not limited to bacterial or viral infection including sepsis, acute pancreatitis, chronic pancreatitis, asthma, allergies, rheumatoid arthritis and adult respiratory distress syndrome.

Autoimmune diseases which may be treated or prevented include, but are not limited to, glomerulonephritis, rheumatoid arthritis (RA), osteoarthritis, systemic lupus erythematosus, scleroderma, chronic thyroiditis, Grave's disease, autoimmune gastritis, insulin-dependent diabetes mellitus (Type I), autoimmune haemolytic anemia, autoimmune neutropenia, thrombocytopenia, atopic dermatitis, chronic active hepatitis, myasthenia gravis, multiple sclerosis, inflammatory bowel disease (IBD), ulcerative colitis, Crohn's disease, chronic obstructive pulmonary disease (COPD), psoriasis, or graft vs. host disease.

Destructive bone disorders which may be treated or prevented include, but are not limited to, osteoporosis, osteoarthritis and multiple myeloma-related bone disorder. Proliferative diseases which may be treated or prevented include, but are not limited to, acute myelogenous leukemia, chronic myelogenous leukemia, metastatic melanoma, Kaposi's sarcoma, and multiple myeloma.

Infectious diseases which may be treated or prevented include, but are not limited to, sepsis, septic shock, and Shigellosis.

Viral diseases which may be treated or prevented include, but are not limited to, acute hepatitis infection (including hepatitis A, hepatitis B and hepatitis C), HIV infection and CMV retinitis.

Degenerative or diseases which may be treated or prevented by the compounds of this invention include, but are not limited to, Alzheimer's disease, Parkinson's disease, cerebral ischemia, and other neurodegenerative diseases.

“p38-mediated conditions” also include ischemia/reperfusion in stroke, heart attacks, myocardial ischemia, organ hypoxia, vascular hyperplasia, cardiac hypertrophy, and thrombin-induced platelet aggregation.

In addition, p38 inhibitors in this invention are also capable of inhibiting the expression of inducible pro-inflammatory proteins such as prostaglandin endoperoxide synthase-2 (PGHS-2), also referred to as cyclooxygenase-2 (COX-2). Therefore, other “p38-mediated conditions” are edema, analgesia, fever and pain, such as neuromuscular pain, headache, cancer pain, dental pain and arthritis pain.

The diseases that may be treated or prevented by the p38 inhibitors of this invention may also be conveniently grouped by the cytokine (IL-1, TNF, IL-6, IL-8) that is believed to be responsible for the disease.

Thus, an IL-1-mediated disease or condition includes rheumatoid arthritis, osteoarthritis, stroke, endotoxemia and/or toxic shock syndrome, inflammatory bowel disease, tuberculosis, artherosclerosis, muscle degeneration, cachexia, psoriatic arthritis, Reiter's syndrome, gout, traumatic arthritis, rubella arthritis, acute synovitis, diabetes, pancreatic B-cell disease and Alzheimer's disease.

TNF-mediated disease or condition includes, rheumatoid arthritis, rheumatoid spondylitis, osteoarthritis, gouty arthritis and other arthritic conditions, sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, fever and myalgias due to infection, cachexia secondary to infection, AIDS, ARC or malignancy, keloid formation, scar tissue formation, Crohn's disease, ulcerative colitis or pyresis. TNF-mediated diseases also include viral infections, such as lentivirus infections, including, but not limited to equine infectious anaemia virus, caprine arthritis virus, visna virus or maedi virus, or retrovirus infections, including feline immunodeficiency virus, bovine immunodeficiency virus, or canine immunodeficiency virus.

IL-8 mediated disease or condition includes diseases characterized by massive neutrophil infiltration, such as psoriasis, inflammatory bowel disease, asthma, cardiac and renal reperfusion injury, adult respiratory distress syndrome, thrombosis and glomerulonephritis.

In addition, the compounds of this infection may be used topically to treat or prevent conditions caused or exacerbated by IL-1 or TNF. Such conditions include inflamed joints, eczema, psoriasis, inflammatory skin conditions such as sunburn, inflammatory eye conditions such as conjunctivitis, pyresis, pain and other conditions associated with inflammation.

In another aspect, the present invention relates to the use of a p38 MAP kinase inhibitor for the preparation of a medicament for treating a subject suffering from a p38-mediated condition, wherein the patient is amenable to the treatment with the p38 MAP kinase inhibitor, if in the subject at least one chromatin remodelling gen is underrepresented and/or at least one pro-inflammatory gene is overrepresented in comparison to a reference sample obtained from a subject not suffering from a p38-mediated condition.

Alternatively, the present invention relates to a method of treating a subject suffering from a p38-mediated condition comprising administering a therapeutically effective amount of a p38 MAP kinase inhibitor to a subject in need thereof, wherein the subject is amenable to the treatment with the p38 MAP kinase inhibitor, if in the patient at least one chromatin remodelling gene is underrepresented and/or at least one pro-inflammatory gene is overrepresented in comparison to a reference sample obtained from a subject not suffering from a p38-mediated condition.

The term “administered” or “administering” means administration of a therapeutically effective dose of a p38 MAP kinase inhibitor to a subject. An “effective amount” is an amount sufficient to effect beneficial or desired clinical results. An effective amount can be administered in one or more administrations. For purposes of this invention, an effective amount is an amount of a p38 MAP kinase inhibitor that is sufficient to ameliorate, stabilize, or delay development of a a p38-mediated condition.

The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

The methods and uses described herein are applicable to both human therapy and veterinary applications. The compounds described herein, in particular, a p38 MAP kinase inhibitor having the desired therapeutic activity may be administered in a physiologically acceptable carrier to a subject, as described herein. Depending upon the manner of introduction, the compounds may be formulated in a variety of ways as discussed below.

The concentration of therapeutically active compound in the formulation may vary from about 0.1-100 wt %. The agents maybe administered alone or in combination with other treatments.

The administration of a p38 MAP kinase inhibitor can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly.

The attending physician and clinical factors will determine the dosage regimen. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors.

Also, the present invention relates to a p38 MAP kinase inhibitor for use in the treatment of a p38-mediated condition in a subject, wherein the subject is amenable to the treatment with the p38 MAP kinase inhibitor, if in the patient at least one chromatin remodelling gene is underrepresented and/or at least one pro-inflammatory gene is overrepresented in comparison to a reference sample obtained from a subject not suffering from a p38-mediated condition.

When used herein, the term “amenable to” means that treating a subject with a p38 MAP kinase inhibitor may have a potentially beneficial effect. Accordingly, a beneficial effect includes, but is not limited to, one or more of the following: alleviation of symptoms, diminishment of extent of a p38-mediated condition, stabilized (i. e., not worsening) state of a p38-mediated condition, preventing spread of a p38-mediated condition, preventing occurrence or recurrence of a p38-mediated condition, delay or slowing of a p38-mediated condition progression, amelioration of the a p38-mediated condition state, and remission (whether partial or total).

The term “treating” or “treatment” includes administration of a p38 MAP kinase, preferably in the form of a medicament, to a subject suffering from a p38-mediated condition for the purpose of ameliorating or improving symptoms of a p38-mediated condition.

In yet another aspect, the present invention relates to the use of a p38 MAP kinase inhibitor for the preparation of a medicament for treating a p38-mediated condition in a subject, wherein the medicament is prepared for an administration pattern comprising administering the medicament to the subject, if in said subject at least one chromatin remodelling gene is underrepresented and/or at least one pro-inflammatory gene is overrepresented in comparison to a reference sample obtained from a subject not suffering from a p38-mediated condition, then discontinuing said administration by means of a continual lack of treatment, if in said subject of at least one chromatin remodelling gene is overrepresented and/or at least one pro-inflammatory gene is underrepresented in comparison to a reference sample obtained from a subject not suffering from a p38-mediated condition and repeating this administration pattern and discontinuance of administration for as long as necessary to achieve a treatment of a p38-mediated condition.

Alternatively, the present invention relates to a method of treating a p38-mediated condition in a subject comprising administering a therapeutically effective amount of a p38 MAP kinase inhibitor, wherein the medicament is for an administration pattern comprising administering a therapeutically effective amount of a p38 MAP kinase inhibitor to the subject, if in said subject at least one chromatin remodelling gene is underrepresented and/or at least one pro-inflammatory gene is overrepresented in comparison to a reference sample obtained from a subject not suffering from a p38-mediated condition, then discontinuing said administration by means of a continual lack of treatment, if in said subject of at least one chromatin remodelling gene is overrepresented and/or at least one pro-inflammatory gene is underrepresented in comparison to a reference sample obtained from a subject not suffering from a p38-mediated condition and repeating this administration pattern and discontinuance of administration for as long as necessary to achieve a treatment of a p38-mediated condition.

Furthermore, the present invention provides a packaged medicament comprising
(a) a p38 MAP kinase inhibitor; and
(b) instructions for use indicating that a subject suffering from a p38-mediated disorder is amenable to the treatment with the p38 MAP kinase inhibitor, if it has been determined whether in said subject at least one chromatin remodelling gene is underrepresented and/or at least one pro-inflammatory gene is overrepresented in comparison to a reference sample obtained from a subject not suffering from a p38-mediated condition.

In addition, the present invention provides a kit for determining whether a subject suffering from a p38-mediated condition is amenable to the treatment with a p38 MAP kinase inhibitor, comprising
(a) means for determining the presence of at least one chromatin remodelling gene and/or at least one pro-inflammatory gene in a sample obtained from said subject; and
(b) instructions for use indicating that a subject suffering from a p38-mediated condition is amenable to the treatment with the p38 MAP kinase inhibitor, if it has been determined whether in said subject at least one chromatin remodelling gene is underrepresented and/or at lest one pro-inflammatory gene is overrepresented in comparison to a reference sample obtained from a subject not suffering from a p38-mediated condition.

In yet another aspect, the present invention provides a kit comprising means for determining the presence of at least one chromatin remodelling gene and/or at least one pro-inflammatory gene in a sample obtained from a subject and instructions for use indicating that determining the presence of at least one chromatin remodelling gene and/or at least one pro-inflammatory gene in a sample obtained from a subject suffering from a p38-mediated condition is indicative whether said subject is amenable to the treatment with a p38 MAP kinase inhibitor, if at least one chromatin remodelling gene is underrepresented and/or at least one pro-inflammatory gene is overrepresented in the sample in comparison to a reference sample obtained from a subject not suffering from a p38-mediated condition.

Each kit includes or consists essentially of at least one probe for a pro-inflammatory gene and/or chromatin remodelling gene as described herein. Reagents or buffers that facilitate the use of the kit can also be included. Any type of probe can be using in the present invention, such as hybridization probes, amplification primers, or antibodies.

In one embodiment, a kit of the present invention includes or consists essentially of at least 1, 2, 3, 4, 5, 6, 7, 8, 9, 10, or more polynucleotide probes or primers. Each probe/primer can hybridize under stringent conditions or nucleic acid array hybridization conditions to a pro-inflammatory gene or chromatin remodelling gene. As used herein, a polynucleotide can hybridize to a gene if the polynucleotide can hybridize to an RNA transcript, or the complement thereof, of the gene.

In another embodiment, a kit of the present invention includes one or more antibodies, each of which is capable of binding to a poiypeptide encoded by a different respective pro-inflammatory gene or chromatin remodelling gene.

The probes employed in the present invention can be either labeled or unlabeled. Labeled probes can be detectable by spectroscopic, photochemical, biochemical, bioelectronic, immunochemical, electrical, optical, chemical, or other suitable means. Exemplary labeling moieties for a probe include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

The kits of the present invention can also have containers containing buffer(s) or reporter means. in addition, the kits can include reagents for conducting positive or negative controls. In one embodiment, the probes employed in the present invention are stably attached to one or more substrate supports. Nucleic acid hybridization or immunoassays can he directly carried out on the substrate support(s). Suitable substrate supports for this purpose include, but are not limited to, glasses, silica, ceramics, nylons, quartz wafers, gels, metals, papers, beads, tubes, fibers, films, membranes, column matrices, or microtiter plate wells. The kits of the present invention may also contain one or more controls, each representing a reference expression level of a prognostic gene detectable by one or more probes contained in the kits.

The Figures show:

FIG. 1: Experimental settings and objectives.

FIG. 2:Downregulation of pro-inflammatory genes in the late stage of LPS-signaling. The “GO” number stands for “gene ontology”. The Gene Ontology project provides a controlled vocabulary to describe gene and gene product attributes in any organism; see http://www.geneontology.org/.

FIG. 3: Quantitative RT-PCR as second independent technique for gene expression changes to conform downregulation of multiple pro-inflammatory genes after long-term treatment with LPS. “FC” means fold change.

FIG. 4: Principal component analysis revealed p38 MAPK to be significantly involved in late-stage LPS-induced gene expression changes. “PD” is the ERK kinase inhibitor PD98059, “GF” is the PKC inhibitor GF109203X and “SB” is the p38 MAP kinase inhibitor SB202190.

FIG. 5: Overrepresentation of chromatin remodelling and nucleosome assembly genes in the group of p38-dependently regulated genes after long-term treatment of monocytes with LPS. The Gene Ontology project provides a controlled vocabulary to describe gene and gene product attributes in any organism; see http://www.geneontology.org/.

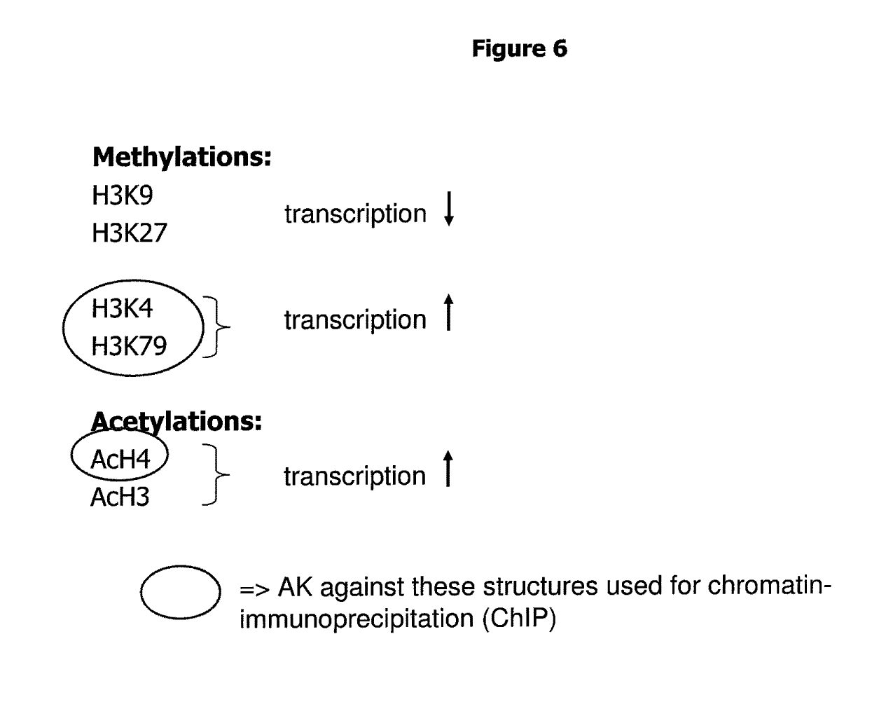

FIG. 6: Role of histone methylation and acetylation-patterns for gene transcription. “H3K9” means the lysine residue at position 9 of histone 3, “H3K27” means the lysine residue at position 27 of histone 3, “H3K4” means the lysine residue at position 4 of histone 3, “H3K79” means the lysine residue at position 79 of histone 3, “AcH4” means acetylation of histone 4 and “AcH3” means acetylation of histone 3.

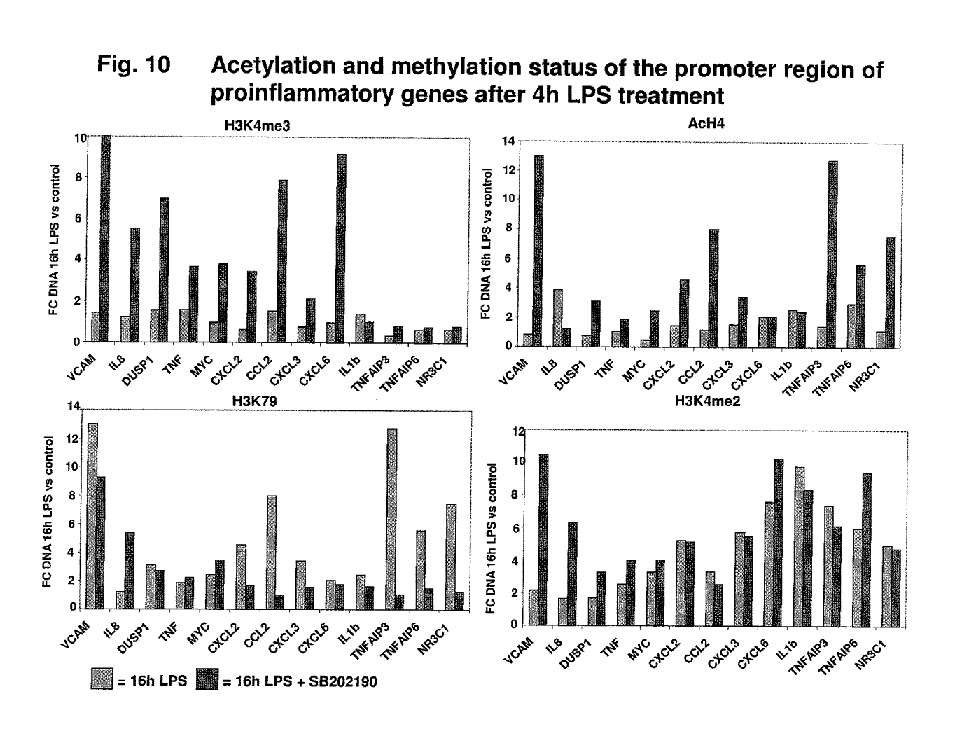

FIG. 7: Histone methylation-and acetylation patterns of LPS-regulated genes in monocytes after 4 hours (FIG. 7A) and 16 hours (FIG. 7B). “H3K4me3” means trimethylation of the lysine residue at position 4 histone 3 and “H3K4me2” means dimethylation of the lysine residue at position 4 histone 3. “FC DNA LPS vs. control” means fold change of the degree of acetylation or methylation of histones in monocytes induced by LPS versus untreated monocytes.

FIG. 8: ChIPs reveal influence of p38 MAP kinase inhibition on epigenetic modifications induced by LPS long-term treatment in monocytes. “FC DNA LPS or LPS+SB vs. control” means fold change of the degree of acetylation or methylation of histones in monocytes induced by LPS or induced by LPS and treated with SB202190 versus untreated monocytes.

FIG. 9: ChIPs reveal an increase in acetylation and tri-methylation of histones located at the promoter region of pro-inflammatory genes after 4 h LPS treatment. The inhibition of p38 MAP kinase does not seem to have an effect on epigenetic modification of pro-inflammatory genes during the first four hours of LPS treatment in monocytes.

FIG. 10: ChIPs reveal no further significant increase in acetylation and tri-methylation of histones located at the promoter region of pro-inflammatory genes after 16 h LPS treatment. However, the inhibition of p38 MAP kinase significantly increased acetylation and tri-methylation of histones located at the promoter region of pro-inflammatory genes, thereby potentially rendering these genes accessible to transcription.

### EXAMPLES

The following examples illustrate the invention. These examples should not be construed as to limit the scope of this invention. The examples are included for purposes of illustration and the present invention is limited only by the claims.

LPS and Reagents

LPS (055:B5, E. coli) and SB202190 were obtained from Sigma-Aldrich (Deisenhof, Germany). PD98059 and GF109203X were purchased from Merck (Darmstadt, Germany).

Cells and Cell Culture

Monocytes were isolated from pooled human buffy coats by Ficoll-Paque and subsequent Percoll (Pharmacia, Freiburg, Germany) density-gradient centrifugation to greater than 90% purity. Monocytes were cultured (1×106 cells/nil) in hydrophobic Teflon bags (Hereus, Hanau, Germany) in McCoy's 5a medium (Biochrom, Berlin, Germany) supplemented with 15% FCS, 2 mM L-glutamine, 200 IU penicillin, 100 μg/ml streptomycin and 1× NEAA (all from Biochrom, Berlin, Germany) and allowed to rest for 18 h prior to stimulation. Monocytes were exposed for 4 and 16 hours to 100 ng/ml LPS in the absence or presence of 10 μM PD98059, 10 μM GF109203X or 10 μm SB202190, respectively, or were left untreated

DNA Microarray Hybridization

Total cellular RNA was isolated from 3 independent experiments with untreated and LPS-stimulated human monocytes in the absence or presence of pharmacological kinase inhibitors (RNeasy kit; Qiagen, Hilden, Germany). RNA preparation, samples preparation and hybridization to Affymetrix Human Genome 133 A Gene Chip arrays were performed according to the manufacturer's instructions (Affymetrix, Santa Clara, Calif.).

Ten micrograms of fragmented cRNA was, together with control cRNAs and grid alignment oligonucleotides, hybridized for 16 hours under constant rotation at 45° C. to Affymetrix Human Genome 133 A Gene Chip arrays (Affymetrix). Arrays were washed and stained using the Gene Chip Fluidics Station 400 (Affymetrix). Fluorescent signals were detected by the HP G2500A Gene Array Scanner (Affymetrix).

Statistical Analysis of Microarray Data

Data were processed by MicroArray Suite (MAS) Software 5.0 (Affymetrix). Signals were scaled to a target intensity of 100 and log transformed. The algorithm applied in the MAS 5.0 software assigned individual average background values to each cell intensity and subtracted it from single raw cell intensities Single raw values were calculated for each probe set (i.e., transcript from the median of discrimination values and their significance was assessed by using a one-sided Wilcoxon rank test). Finally, the whole algorithm resulted in the determination of reasonable array zone-related background thresholds and the assignment of detection cells, “absent” or “present”, to each transcript.

While the MAS 5.0 software only allows comparison of 2 individuals data sets, we further studied the Affymetrix intensity files with the Expressionist Suite software package (GeneData, Basel, Switzerland) for a more sophisticated statistical analysis. A chain of statistical methods is applied as a mining tool to identify genes significantly regulated in multiple independent experiments. The software package includes the Expressionist Refiner v3.0.4 which permits a global chip quality control with detection and masking of outliers and array defects, fluorescence gradient correction, and variance regularization. For final identification of significant gene expression difference we used Expressionist Analyst v4.0.5. First, data were normalized to a logarithmic mean of 180 in all experiments. We filtered for genes with valid values (i.e., an expression over background or a detection call “on”) in more than 50% of the respective experimental group. Finally, n-fold changes were determined and screened for their significance by applying a t test. We retained only genes with a fold change of at least 2.0 or no more than −2.0 and a p value of less than 0.05. To consider “on/off” phenomena in gene expression we defined genes that were present in each of the 3 individual experiments of the stimulation (or inhibition) groups and absent in all 3 experiments of the control group (i.e., genes with an “on/off” ratio of 3:0, as significantly regulated). To differentiate on/off phenomena occurring around the background threshold from significant on/off phenomena we used normalized Analyst raw data and performed n-fold determinations and student t test calculations without applying the valid value filter. Being aware of the low significance of intensity ratios at low intensity levels we only defined regulations with a high fold change of at least 5 and a p value of less than 0.05 as relevant, since “on” then means an expression level high over background.

We applied principal component analyses (PCAs) to reduce mathematically the dimensionality of the entire spectrums of gene expression values of a microarray experiment to 3 components (Alter et al. 2000, Proc. Natl. Acad. Sci. U.S.A (97):10111-6).

PCA is a bioinformatic tool that allows the description of gene profiles virtually as vector clouds in a three-dimensional vector space.

To identify functional categories of genes that are overrepresented in the data sets of regulated genes we first assigned Gene Ontology (GO) annotations to every probe set spotted on the Affymetrix 133 Plus 2.0 Array and compared it with the distribution of GO annotations in the gene group of interest applying Fisher's exact test. In case of genes that are represented by two or more probe sets only one transcript was taken into account in order to avoid potential bias.

Real-Time RT-PCR

For real-time RT-PCR, RNA from every experimental group was analyzed in dublicate, cDNA was synthesized from 4 μg of total RNA using SuperScript II RNase H-reverse transcriptase (Invitrogen, Carlsbad, Calif.). Primers were designed using the Primer Express software package (Applied Biosystems, Foster City, Calif.) and obtained from MWG Biotech (Ebersberg, Germany).The primers used for PCR analysis were as follows:

| Gene | Forward | Reverse |
|---|---|---|
| GAPDH | AGGTGGTCTCCTCT | TGTAGGCTTCAGA |
| | GACTTCAACA | CGCACGAC |
| | (SEQ ID NO: 1) | (SEQ ID NO: 2) |
| CCL20 | TTCTGGAATGGAA | ACCCTCCATGA |
| | TTGGACATAGC | TGTGCAAGTG |
| | (SEQ ID NO: 3) | (SEQ ID NO: 4) |
| IL6 | ACCACCGGAAGG | TTCACACAGAGCTGC |
| | AACCATTC | AGAAATCA |
| | (SEQ ID NO: 5) | (SEQ ID NO: 6) |
| CXCL2 | ACATCCAAAGTGT | AAGCTTTCTGCCC |
| | GAAGGTGAAGTC | ATTCTTGAGT |
| | (SEQ ID NO: 7) | (SEQ ID NO: 8) |
| CCL2 | TCGCCTCCAGC | TTGCATCTGGC |
| | ATGAAAGTC | TGAGCGAG |
| | (SEQ ID NO: 9) | (SEQ ID NO: 10) |
| CXCL3 | AGCGTATCATTGA | TCCCTTTCCAGC |
| | CACTTCCTGC | TGTCCCTAG |
| | (SEQ ID NO: 11) | (SEQ ID NO: 12) |
| CXCL6 | CGATTGGTAAACT | TCCGGGTCCAGA |
| | GCAGGTGTTC | CAAACTTG |
| | (SEQ ID NO: 13) | (SEQ ID NO: 14) |
| CD80 | CTGCTTTGCCCC | CAGATCTTTTCAG |
| | AAGATGC | CCCCTTGC |
| | (SEQ ID NO: 15) | (SEQ ID NO: 16) |
| TNF | CTTCTCGAACCC | TGAGGTACAGGCC |
| | CGAGTGAC | CTCTGATG |
| | (SEQ ID NO: 17) | (SEQ ID NO: 18) |
| IL1B | GCGGCCAGGATAT | TCCACATTCAGCAC |
| | AACTGACTTC | AGGACTCTC |
| | (SEQ ID NO: 19) | (SEQ ID NO: 20) |
| IL7R | AGGAGCCAATGA | CTGGCGGTAAGC |
| | CTTTGTGGTG | TACATCGTG |
| | (SEQ ID NO: 21) | (SEQ ID NO: 22) |
| CCL5 | GCCTGTTTCTGCT | TGCTCGTCGTGG |
| | TGCTCTTGT | TCAGAATCT |
| | (SEQ ID NO: 23) | (SEQ ID NO: 24) |

Real-time RT-PCR was performed using the QuantiTec SYBR Green PCR kit (Qiagen) under conditions as described earlier. Data were acquired with the ABI PRISM 7900 (Applied Biosystems). Gene expression was normalized with respect to the endogenous housekeeping control gene glyceraldehyde phosphate dehydrogenase (GAPDH) The relative expression of respective genes was calculated by using the comparative threshold cycle (CT) method as described (Liu and Saint 2002, Anal. Biochem.(302):52-9).

Chromatin Immunoprecipitation (ChIP)

After stimulation, monocytes were formalin-fixed, washed, lysed, and sonicated. DNA-protein complexes were immunoprecipitated with polyclonal histone-antibodies against H3K4 (abcam, Cambridge, UK), H3K79 and AcH4 (both upstate, Lake Placid, N.Y.). Precipitates were extracted by protein NG-Sepharose beads (Santa Cruz Biotechnology), washed, and digested with proteinase K. After reversing the cross-link between DNA and protein, DNA was extracted and amplified with primers hybridizing to promoter regions of LPS-regulated proinflammatory and immunoregulatory genes as VCAM, IL8, DUSP1, TNF and MYC. PCR products were processed by using the QuantiTect SYBR Green PCR kit to perform quantitative RT-PCRs employing the ABI PRISM 7900 (Applied Biosystems).

Promoter-primers used were as follows:

| Gene | Forward | Reverse |
|---|---|---|
| GAPDH | GGAGGTTGTAGT | TGAAGCAGCTCA |
| | GAGCCGAAAT | ACGCAGTT |
| | (SEQ ID NO: 25) | (SEQ ID NO: 26) |
| VCAM | ACTTTTTTCCCT | TTCAGCTCCTGAA |
| | GGCTCTGCC | GCCAGTGA |
| | (SEQ ID NO: 27) | (SEQ ID NO: 28) |
| IL8 | TCTTCCTCTATTGA | TCCAGTGGAGGCA |
| | AGCCCTCCTA | TAAGAGCA |
| | (SEQ ID NO: 29) | (SEQ ID NO: 30) |
| DUSP1 | CGAGGGTTGTGGC | GGGTTTGGCGTTT |
| | CGGCTTCTGTT | GGAGGGGTGAG |
| | (SEQ ID NO: 31) | (SEQ ID NO: 32) |
| TNF | AACCGAGACAGA | AGAATCATTCAA |
| | AGGTGCAGG | CCAGCGGAA |
| | (SEQ ID NO: 33) | (SEQ ID NO: 34) |
| MYC | AAGGGCAGGGC | GCGAGTTAGATAA |
| | TTCTCAGAG | AGCCCCGAA |
| | (SEQ ID NO: 35) | (SEQ ID NO: 36) |

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

1. A method for determining whether therapy with a p38 MAP kinase inhibitor is potentially beneficial or potentially contraindicated for a subject suffering from a p38-mediated condition comprising measuring in a sample obtained from the subject the presence of at least one chromatin remodelling gene and/or of at least one pro-inflammatory gene, wherein the treatment is potentially beneficial for the subject, if at least one chromatin remodelling gene is underrepresented and/or at least one pro-inflammatory gene is overrepresented in comparison to a reference sample obtained from a subject not suffering from a p38-mediated condition; or wherein the treatment is potentially contraindicated for the subject, if at least one chromatin remodelling gene is overrepresented and/or at least one proinflammatory gene is underrepresented in comparison to a reference sample obtained from a subject not suffering from a p38-mediated condition.

2. The method of claim 1, wherein said chromatin remodelling gene are involved in epigenetic silencing.

3. The method of claim 1, wherein said chromatin remodelling gene encodes a protein involved in epigenetic silencing.

4. The method of claim 1, wherein said pro-inflammatory gene encodes a cytokine.

5. The method of claim 1, wherein the sample comprises monocytes.

6. The method of claim 1, wherein the subject is a mammal, preferably a human.

7. The method of claim 1, wherein the p38-mediated condition is selected from the group consisting of inflammatory diseases, autoimmune diseases, destructive bone disorders, proliferative disorders, infectious disease, viral diseases, and neurodegenerative diseases.

8. Use of a p38 MAP kinase inhibitor for the preparation of a medicament for treating a subject suffering from a p38-mediated condition, wherein the patient is amendable to the treatment with the p38 MAP kinase inhibitor, if in the subject at least one chromatin remodelling gene is underrepresented and/or at least one proinflammatory gene is overrepresented in comparison to a reference sample obtained from a subject not suffering from a p38-mediated condition.

9. A p38 MAP kinase inhibitor for use in the treatment of a p38-mediated condition in a subject, wherein the subject is amenable to the treatment with the p38 MAP kinase inhibitor, if in the patient at least one chromatin remodelling gene is underrepresented and/or at least one pro-inflammatory gene is overrepresented in comparison to a reference sample obtained from a subject not suffering from a p38-mediated condition.

10. Use of a p38 MAP kinase inhibitor for the preparation of a medicament for treating a p38-mediated condition in a subject, wherein the medicament is prepared for an administration pattern comprising administering the medicament to the subject, if in said subject at least one chromatin remodelling gene is underrepresented and/or at least one pro-inflammatory gene is overrepresented in comparison to a reference sample obtained from a subject not suffering from a p38-mediated condition, then discontinuing said administration by means of a continual lack of treatment, if in said subject of at least one chromatin remodelling gene is overrepresented and/or at least one pro-inflammatory gene is underrepresented in comparison to a reference sample obtained from a subject not suffering from a p38-mediated condition and repeating this administration pattern and discontinuance of administration for as long as necessary to achieve a treatment of a p38-mediated condition.

11. A packaged medicament comprising
(a) a p38 MAP kinase inhibitor; and
(b) instructions for use indicating that a subject suffering from a p38-mediated disorder is amenable to the treatment with the p38 MAP kinase inhibitor, if it has been determined whether in said subject at least one chromatin remodelling gene is underrepresented and/or at least one pro-inflammatory gene is overrepresented in comparison to a reference sample obtained from a subject not suffering from a p38-mediated condition.

12. A kit for determining whether a subject suffering from a p38-mediated condition is amenable to the treatment with a p38 MAP kinase inhibitor, comprising
(a) means for determining the presence of at least one chromatin remodelling gene and/or at least one pro-inflammatory gene in a sample obtained from said subject; and
(b) instructions for use indicating that a subject suffering from a p38-mediated condition is amenable to the treatment with the p38 MAP kinase inhibitor, if it has been determined whether in said subject at least one chromatin remodelling gene is underrepresented and/or at least one pro-inflammatory gene is overrepresented in comparison to a reference sample obtained from a subject not suffering from a p38-mediated condition.

13. A kit comprising means for determining the presence of at least one chromatin remodelling gene and/or at least one pro-inflammatory gene in a sample obtained from a subject and instructions for use indicating that determining the presence of at least one chromatin remodelling gene and/or at least one pro-inflammatory gene in a sample obtained from a subject suffering from a p38-mediated condition is indicative whether said subject is amenable to the treatment with a p38 MAP kinase inhibitor, if at least one chromatin remodelling gene is underrepresented and/or at least one pro-inflammatory gene is overrepresented in the sample in comparison to a reference sample obtained from a subject not suffering from a p38-mediated condition.


SEQUENCE LISTING (36 sequences)

SEQ ID NO: 1 - DNA, 24 bp, Artificial Sequence
  PCR primer for human GAPDH
aggtggtctcctctgacttcaaca

SEQ ID NO: 2 - DNA, 21 bp, Artificial Sequence
  PCR primer for human GAPDH
tgtaggcttcagacgcacgac

SEQ ID NO: 3 - DNA, 24 bp, Artificial Sequence
  PCR primer for human CCL20
ttctggaatggaattggacatagc

SEQ ID NO: 4 - DNA, 21 bp, Artificial Sequence
  PCR primer for human CCL20
accctccatgatgtgcaagtg

SEQ ID NO: 5 - DNA, 20 bp, Artificial Sequence
  PCR primer for human IL6
accaccggaaggaaccattc

SEQ ID NO: 6 - DNA, 23 bp, Artificial Sequence
  PCR primer for human IL6
ttcacacagagctgcagaaatca

SEQ ID NO: 7 - DNA, 25 bp, Artificial Sequence
  PCR primer for human CXCL2
acatccaaagtgtgaaggtgaagtc

SEQ ID NO: 8 - DNA, 23 bp, Artificial Sequence
  PCR primer for human CXCL2
aagctttctgcccattcttgagt

SEQ ID NO: 9 - DNA, 20 bp, Artificial Sequence
  PCR primer for human CCL2
tcgcctccagcatgaaagtc

SEQ ID NO: 10 - DNA, 19 bp, Artificial Sequence
  PCR primer for human CCL2
ttgcatctggctgagcgag

SEQ ID NO: 11 - DNA, 23 bp, Artificial Sequence
  PCR primer for human CXCL3
agcgtatcattgacacttcctgc

SEQ ID NO: 12 - DNA, 21 bp, Artificial Sequence
  PCR primer for human CXCL3
tccctttccagctgtccctag

SEQ ID NO: 13 - DNA, 23 bp, Artificial Sequence
  PCR primer for human CXCL6
cgattggtaaactgcaggtgttc

SEQ ID NO: 14 - DNA, 20 bp, Artificial Sequence
  PCR primer for human CXCL6
tccgggtccagacaaacttg

SEQ ID NO: 15 - DNA, 19 bp, Artificial Sequence
  PCR primer for human CD80
ctgctttgccccaagatgc

SEQ ID NO: 16 - DNA, 21 bp, Artificial Sequence
  PCR primer for human CD80
cagatcttttcagccccttgc

SEQ ID NO: 17 - DNA, 20 bp, Artificial Sequence
  PCR primer for human TNF
cttctcgaaccccgagtgac

SEQ ID NO: 18 - DNA, 21 bp, Artificial Sequence
  PCR primer for human TNF
tgaggtacaggccctctgatg

SEQ ID NO: 19 - DNA, 23 bp, Artificial Sequence
  PCR primer for human IL1B
gcggccaggatataactgacttc

SEQ ID NO: 20 - DNA, 23 bp, Artificial Sequence
  PCR primer for human IL1B
tccacattcagcacaggactctc

SEQ ID NO: 21 - DNA, 22 bp, Artificial Sequence
  PCR primer for human IL7R
aggagccaatgactttgtggtg

SEQ ID NO: 22 - DNA, 21 bp, Artificial Sequence
  PCR primer for human IL7R
ctggcggtaagctacatcgtg

SEQ ID NO: 23 - DNA, 22 bp, Artificial Sequence
  PCR primer for human CCL5
gcctgtttctgcttgctcttgt

SEQ ID NO: 24 - DNA, 21 bp, Artificial Sequence
  PCR primer for human CCL5
tgctcgtcgtggtcagaatct

SEQ ID NO: 25 - DNA, 22 bp, Artificial Sequence
  Oligonucleotide for amplifying GAPDH
ggaggttgtagtgagccgaaat

SEQ ID NO: 26 - DNA, 20 bp, Artificial Sequence
  Oligonucleotide for amplifying GAPDH
tgaagcagctcaacgcagtt

SEQ ID NO: 27 - DNA, 21 bp, Artificial Sequence
  Oligonucleotide for amplifying VCAM
acttttttccctggctctgcc

SEQ ID NO: 28 - DNA, 21 bp, Artificial Sequence
  Oligonucleotide for amplifying VCAM
ttcagctcctgaagccagtga

SEQ ID NO: 29 - DNA, 24 bp, Artificial Sequence
  Oligonucleotide for amplifying IL8
tcttcctctattgaagccctccta

SEQ ID NO: 30 - DNA, 21 bp, Artificial Sequence
  Oligonucleotide for amplifying IL8
tccagtggaggcataagagca

SEQ ID NO: 31 - DNA, 24 bp, Artificial Sequence
  Oligonucleotide for amplifying DUSP1
cgagggttgtggccggcttctgtt

SEQ ID NO: 32 - DNA, 24 bp, Artificial Sequence
  Oligonucleotide for amplifying DUSP1
gggtttggcgtttggaggggtgag

SEQ ID NO: 33 - DNA, 21 bp, Artificial Sequence
  Oligonucleotide for amplifying TNF
aaccgagacagaaggtgcagg

SEQ ID NO: 34 - DNA, 21 bp, Artificial Sequence
  Oligonucleotide for amplifying TNF
agaatcattcaaccagcggaa

SEQ ID NO: 35 - DNA, 20 bp, Artificial Sequence
  Oligonucleotide for amplifying MYC
aagggcagggcttctcagag

SEQ ID NO: 36 - DNA, 22 bp, Artificial Sequence
  Oligonucleotide for amplifying MYC
gcgagttagataaagccccgaa